(19) United States
(12) Patent Application Publication
(10) Pub. No.: US 2008/0097294 A1
(43) Pub. Date: Apr. 24, 2008

(54) Occlusive guidewire system having an ergonomic handheld control mechanism prepackaged in a pressurized gaseous environment and a compatible prepackaged torqueable kink-resistant guidewire with distal occlusive balloon

(75) Inventors: Richard Russell Prather, St. Michael, MN (US); Leif Erik Leirfallom, Minneapolis, MN (US); Eric Joel Thor, Arden Hills, MN (US); Michael John Bonnette, Minneapolis, MN (US); Robert Alan Lambert, Lake Elmo, MN (US); Laszlo Trent Farago, Hudson, WI (US)

Correspondence Address: HUGH D JAEGER, P.A., P.O. BOX 672, WAYZATA, MN, 55391-0672, US

(73) Assignee: Possis Medical, Inc.

(21) Appl. No.: 11/581,613

(22) Filed: Oct. 16, 2006

Related U.S. Application Data
(60) Provisional application No. 60/775,259, filed on Feb. 21, 2006.
(60) Provisional application No. 60/798,965, filed on May 9, 2006.
(60) Provisional application No. 60/799,246, filed on May 10, 2006.
(60) Provisional application No. 60/799,498, filed on May 11, 2006.
(60) Provisional application No. 60/801,173, filed on May 17, 2006.

(51) Int. Cl. A61M 31/00; B65B 31/04

(52) U.S. Cl. 604/95.04; 53/403

(57) ABSTRACT

An occlusive guidewire system having an ergonomic handheld control mechanism prepackaged in a pressurized gaseous environment and a compatible prepackaged torqueable kink-resistant guidewire with distal occlusive balloon. Convenient structure and overall mechanism for operation of a torqueable kink-resistant guidewire with a distal occlusive balloon, including evacuation and inflation control of the distal occlusive balloon, and sealing and severing of a crimpable inflation tube which is in communication with the occlusive balloon. Torqueable kink-resistant guidewires include centrally located structure which imparts robustness to the torqueable kink-resistant guidewires. An inflation lumen aligns within the torqueable kink-resistant guidewires for inflation of the occlusive balloon.

### CROSS REFERENCES TO RELATED APPLICATIONS

This patent application is related to application Ser. No. 11/217,545 entitled “Occlusive Guidewire System Having an Ergonomic Handheld Control Mechanism and Torqueable Kink-Resistant Guidewire” filed on Sep. 01, 2005. This patent application is also related to application Ser. No. 10/838,464 entitled “Gas Inflation/Evacuation System and Sealing System Incorporating a Compression Sealing Mechanism for Guidewire Assembly Having Occlusive Device” filed on May 04, 2004, and application Ser. No. 10/838,468 entitled “Guidewire Assembly Including a Repeatably Inflatable Occlusive Balloon on a Guidewire Ensheathed with a Spiral Coil” filed on May 04, 2004, both of which are continuations-in-part of application Ser. Nos. 10/012,903, 10/012,891 and 10/007,788 all filed on Nov. 06, 2001. This patent application is also closely related to patent application Ser. No. 10/930,528 entitled “Low Pierce Force Needle Port” filed on Aug. 31, 2004.

This application claims benefit from earlier filed U.S. Provisional Applications, as follows: Appl. No. 60/775,259 entitled “Catheter Balloon” filed Feb. 21, 2006; Appl. No. 60/798,965 entitled “Catheter Balloon” filed May 09, 2006; Appl. No. 60/799,246 entitled “Catheter Packaging System” filed May 10, 2006; Appl. No. 60/799,498 entitled “Seal System” filed May 11, 2006; and Appl. No. 60/801,173 entitled “Guidewire System” filed May 17, 2006, all of which are hereby incorporated into this application by reference as if fully set forth herein.

### BACKGROUND OF THE INVENTION

1. Field of the Invention

The present invention relates to medical devices, but more directly relates to a unique gaseous environment prepackaging method encompassing an improved ergonomic handheld control mechanism employed for use with separately packaged torqueable kink-resistant guidewires for temporary gaseous substance inflation of a guidewire mounted occlusive balloon in the vasculature during surgical procedures.

2. Description of the Prior Art

Prior art packaging of medical devices in a sterile condition often was faulty due to gas pressure loss caused by irradiation of polymers in the presence of carbon dioxide included within the encompassing packaging enclosure, as evidenced by undesirable collapsing of the enclosure about the medical device. Packaging methods include, but are not limited to, packaging at near ambient pressure in both rigid and flexible containers that may develop leaks upon their collapse in a vacuum state. The principal disadvantage of these methods is the loss of critical sterility and special gaseous environment necessary to the safety and function of the contained material, as in the case of medical devices and the like. The present invention overcomes the inadequacies of the prior art packaging methods by providing a rigid container and method of overpressurizing of carbon dioxide gas to account for the loss of partial pressure that occurs through reaction between the polymer, such as polycarbonate, and the carbon dioxide gas therewithin in the process of gamma or other suitable sterilizing radiation.

Prior art devices included an inflation tube sealing mechanism incorporated with a closely coupled and intimately engaged compression sealing mechanism used to seal a crimpable inflation tube at the proximal end of a torqueable kink-resistant guidewire. Generally, a seal included in the compression sealing mechanism, through which a crimpable inflation tube of a torqueable kink-resistant guidewire passes for the purpose of conducting a flow of gas or liquid through it, may not properly seal after gamma sterilization or may not perform after being pierced by a number of randomly inserted crimpable inflation tubes. When gamma sterilization is applied to a silicone seal, its elongation is compromised and the silicone may become embrittled and tear a hole that does not seal around the crimpable inflation tubes. Then, when pierced by multiple crimpable inflation tubes, the crimpable inflation tubes may penetrate the seal next to each other and result in additional tearing and, hence, leaking. The present invention overcomes the inadequacies of the prior art devices by providing a multiple guide cell seal which is of a composition that is of very soft silicone so that conformance thereof to the sides of the crimpable inflation tubes seals pressure or vacuum; which has redundant multiple and adjacent seals to reduce the probability that two crimpable inflation tubes next to each other will create a leak; which after gamma sterilization the very soft silicone remains soft even though harder than before sterilization (continues to cross-link, but does not get so brittle that it does not seal); and which includes adjacent and intersecting sloping sides which are steep enough to guide or deflect an incoming crimpable inflation tube away from already engaged crimpable inflation tubes into adjacent sloping guide cells.

Prior art devices have incorporated balloons to provide for temporary occlusions in the vasculature, whereby an inflatable balloon attached to the distal end of a guidewire having an internal inflation lumen is inflated. Such devices are useful during cross stream thrombectomy procedures where the guidewire having an internal inflation lumen can be used as an ordinary guidewire. Alternatively, such devices can also be useful to prevent downstream distribution of lysins beyond a region of thrombus or other undesirable buildup. The structure of the prior art devices incorporated to operate a guidewire having an internal inflation lumen often included a collection of multiple components coupled together to provide for bulky or cumbersome connection of multiple tubes, valves, syringes, connectors and other associated components. Often, the assembled collection of components proved to be of an unwieldy nature and was awkward to use. In addition to the user unfriendly aspects of the prior art devices, other problems were encountered when aligning the guidewire having an internal inflation lumen in the vasculature. Due to the small size of the guidewire with a lumen and due to the lack of robustness, undesirable kinking and bending of the guidewire occurred when positioning the guidewire along the vasculature. Such undesirable kinking and bending also occurred when the guidewire having a lumen was torqued or twisted about its flexible longitudinal axis in order to steer a flexible tip along tortuous paths of the vasculature. The present invention overcomes the inadequacies of the prior art by providing an occlusive guidewire system having an ergonomic handheld control mechanism prepackaged in a pressurized gaseous environment and a compatible prepackaged torqueable kink-resistant occlusive guidewire with distal occlusive balloon.

Prior art devices included hubless torqueable kink-resistant guidewires having inflatable balloons functioning as an occluder at the distal portion thereof. Maintaining a minimum deflated (crossing) profile has always been a concern in order to provide a minimum crossing size for passage through other associated devices. The present invention overcomes the inadequacies of the prior art devices by providing a novel balloon attachment and configuration methods promoting a minimum crossing profile.

### SUMMARY OF THE INVENTION

The general purpose of the present invention is to provide an occlusive guidewire system having an ergonomic handheld control mechanism prepackaged in a pressurized gaseous environment and a compatible prepackaged torqueable kink-resistant guidewire with distal occlusive balloon.

The instant invention is a system and includes components comprising a structure for enclosing other components in a gaseous environment, components actually enclosed in the enclosing structure having a gaseous environment, and other prepackaged components which do not derive benefit from being enclosed in a gaseous environment, but which are used in conjunction with the enclosed components.

A canister and a canister lid are provided as a sealable unit for containment of a handheld control mechanism and packaging and informational material. The canister and canister lid comprise an airtight package capable of retaining a suitable inflation gaseous substance, such as, but not limited to, carbon dioxide. The canister and canister lid sealingly surround the handheld control mechanism to contain pressurized carbon dioxide.

The handheld control mechanism incorporates, but is not limited to, an upper housing half and a lower housing half whereat a plurality of components included in an internal mechanism assembly secure to, within or thereabout, including an inflation syringe, an evacuation syringe, an inline inflation control valve and actuator button, an access hole leading to a low pierce force needle injection port, an inline evacuation control valve and actuator button, an inflation tube sealing mechanism, a compression sealing mechanism, a pressure gauge, and a four-port infusion “y”. The handheld control mechanism is ergonomically designed and shaped to provide for easy and efficient operation during medical procedures, as well as to provide convenient housing of components. Torqueable and kink-resistant guidewires of different sizes and styles, as described herein, the proximal ends of which can be accommodated by the handheld control mechanism, include an inflatable occlusive device preferably in the form of a balloon which is distally located thereupon. This invention relates to the balloon occlusive/distal protection guidewire technology and functions like other guidewires having distally located inflatable occlusive devices that are already being produced, i.e., CO₂ filled balloons that utilize a special crimping/inflation device to seal them and make them hubless to be used as an ordinary guidewire. The use of CO₂ gas allows for rapid inflation or deflation of an occlusive balloon as opposed to liquid inflative devices having slower inflation or deflation response times. The access hole in a lower housing half of the handheld control mechanism provides ready access for injection of carbon dioxide or other suitable gas into an inflation syringe central to the handheld control mechanism. This invention relates to an enhancement to the existing technology.

A multiple guide cell seal is incorporated within the inflation tube sealing mechanism having geometric shapes incorporated to direct or steer multiple crimpable inflation tubes one at a time away from each other, thereby reducing the probability of an additional crimpable inflation tube sliding directly along another previously placed crimpable inflation tube and tearing or enlarging one or more of the adjacent silicone seals and the multiple guide cell seal, thereby increasing the ability to obtain a favorable puncturing outcome whereby separate puncture hole sets for each of the crimpable inflation tubes are formed being spaced across the seals.

One torqueable kink-resistant guidewire includes a balloon which is inflatable and which is deflatable and a flexible tip located at or near a distal location along the guidewire. The torqueable kink-resistant guidewire consists primarily of components, in proximal to distal connected order, including a crimpable inflation tube, a coaxially arranged supporting inflation tube and free-floating primary inflation tube, an inflation tube, a balloon attached to and aligned over and about the inflation tube, a spring coil aligned over and secured about the inflation tube proximal to the balloon, a spring coil aligned over and secured about the inflation tube distal to the balloon, and a flexible guidewire tip. The coaxial arrangement of the supporting inflation tube and the primary inflation tube provides the kink resistance to the device due to its flexible properties and the torqueability of the device since it transfers proximal rotational force to the distal section in a one-to-one fashion because of the physical structure of the device. Torque response is a necessity in guiding the guidewire through tortuous vasculature. The inflation tube, in involvement with the coaxially arranged supporting inflation tube and free-floating primary inflation tube, transfers inflation gas, preferably CO₂ from the inflation structure in the handheld control mechanism to the balloon. The crimpable inflation tube or the coaxially arranged supporting inflation tube and free-floating primary inflation tube could be metal, plastic or composite. The crimpable inflation tube is designed to have crimpable attributes such that portions thereof can be repeatably sealed via the inflation tube sealing mechanism, a special crimping device contained within the handheld control mechanism. The sealed proximal end can be removed from the inflation tube sealing mechanism of the handheld control mechanism so that the wire can be used like an ordinary guidewire as a hubless system. The crimpable inflation tube needs to be of a specific dimension, material and hardness to be compatible with the inflation tube sealing mechanism, such as metal with a medium hardness. The balloon can be made from many different materials that may be noncompliant, semi-compliant, or compliant, such as, but not restricted to, Pellethane 2363 80AE, a polyurethane, silicone, Pebax, or other polyurethanes. The purpose of the balloon is to occlude flow to prevent distal embolization of particles (which cause more damage), to minimize hemolytic components or drugs from flowing throughout the body, to contain other agents, to center another device within the vessel, or to provide for an isolated environment within a vessel. The torqueable kink-resistant guidewire can be coated, such as with a hydrophilic coating with the exception of the inflation balloon, the flexible wire coil spring and the crimpable inflation tube, to improve trackability or compatibility with other interventional devices, or uncoated depending on the polymer used and the use and required performance of the guidewire. The polymer can be any type of polymer, but most preferably one that is flexible enough to allow for appropriate guidewire structure, and one that is rigid enough to aid in torqueability. It is also preferred to have this polymer loaded with a radiopaque material, such as tungsten or BaSO₄ (barium sulfate), to improve the visibility of the device under normal fluoroscopy. The guidewire tip usually consists of a core and an outer coil. The design of the guidewire tip is important such that it allows the device to be steered and placed in the damaged vasculature.

Another torqueable kink-resistant guidewire includes a balloon which is inflatable and which is deflatable and a flexible tip located at or near a distal location along the guidewire. The torqueable kink-resistant guidewire consists primarily of components in proximal to distal connected order, including a crimpable inflation tube continuous with a proximally located inflation tube, a receptacle at the distal end of the proximally located inflation tube which accommodates the proximal end of a centrally located inflation tube to form a low profile joint, a proximally located spring coil aligned over and about the distal end of the centrally located inflation tube just proximal to a plurality of inflation orifices, a distally located spring coil aligned over and about the distal end of the centrally located inflation tube just distal to the plurality of inflation orifices, a balloon aligned over and about the inflation orifices where the proximal balloon neck and the distal balloon neck secure over and about the distal end of the proximally located spring coil and the proximally located end of the distally located spring coil, respectively, and a floppy tip core connected to the distal end of the centrally located inflation tube extending distally along the interior of the distally located spring coil to terminate at a rounded distal tip.

According to one or more embodiments of the present invention, there is provided an occlusive guidewire system having an ergonomic handheld control mechanism prepackaged in a pressurized gaseous environment and compatible prepackaged torqueable kink-resistant guidewire with distal occlusive balloon. The ergonomic handheld control mechanism and a gaseous substance, such as, but not limited to, carbon dioxide, are contained within and hermetically sealed in a surrounding canister and removably attached canister lid. A sealed pouch includes a transportation coil which protectively houses one of several styles of torqueable kink-resistant guidewires.

One significant aspect and feature of the present invention is an occlusive guidewire system having a pressurized gaseous environment, such as, but not limited to, the use of carbon dioxide contained by a canister or other suitable enclosure.

Another significant aspect and feature of the present invention is the use of a pressurized gaseous environment, such as, but not limited to, pressurized carbon dioxide, to interact with inflation structure, thereby counteracting pressure loss occurring from irridation of polymers in the presence of carbon dioxide.

One significant aspect and feature of the present invention is an occlusive guidewire system having an ergonomic handheld control mechanism and torqueable kink-resistant guidewire.

Another significant aspect and feature of the present invention is an ergonomic handheld control mechanism which conveniently contains mounted components essential to the operation of a torqueable kink-resistant guidewire assembled for minimizing problems encountered in freeform and unrestrained arrangements where components are not secured for efficient and useful operation thereof.

Another significant aspect and feature of the present invention is an ergonomic handheld control mechanism having simple lineally actuated control valves being readily accessible by the operator as opposed to rotary valves incorporated in other arrangements.

Yet another significant aspect and feature of the present invention is an ergonomic handheld control mechanism having lineally actuated control valves aligned within arcuate recesses to allow only wanted actuation and to prevent inadvertent control valve actuation.

Still another significant aspect and feature of the present invention is an ergonomic handheld control mechanism having a built-in and mounted pressure gauge.

Another significant aspect and feature of the present invention is an ergonomic handheld control mechanism having a built-in and mounted inflation tube sealing mechanism and a compression sealing mechanism.

A further significant aspect and feature of the present invention is an ergonomic handheld control mechanism having a built-in and mounted evacuation syringe and inflation syringe.

Another significant aspect and feature of the present invention is an ergonomic handheld control mechanism having built-in and mounted check valves.

Another significant aspect and feature of the present invention is the use of a multiple guide cell seal in an inflation tube sealing mechanism in order to promote orderly sealing accommodation of multiple punctures by more than one crimpable inflation tube.

In addition to the above, significant aspects and features of the present invention also involve a torqueable kink-resistant guidewire having common traits, features and the like, wherein:

1. the torqueable kink-resistant guidewire employs a distal occlusive balloon which is stretchably mounted to facilitate a minimum crossing profile;

2. the torqueable kink-resistant guidewire with distal occlusive balloon employs crimpable/sealable structure that allows other devices to be passed over it while the distal occlusive balloon is inflated, i.e., a hubless balloon guidewire;

3. the torqueable kink-resistant guidewire with distal occlusive balloon uses gas (preferably CO₂ but could be argon or helium) as an inflation medium;

4. the torqueable kink-resistant guidewire with distal occlusive balloon is radiopaque, such as by the use of a tungsten or barium sulfate filled polyurethane or other suitable material;

5. portions of the torqueable kink-resistant guidewire with distal occlusive balloon can be coated with a hydrophilic material to give ultra lubricity;

6. the torqueable kink-resistant guidewire with distal occlusive balloon uses a conduit or inflation lumen along the entire length and ending under the distal occlusive balloon to transfer pressurized inflation gas to the balloon which is constructed by any of the means described above;

7. the torqueable kink-resistant guidewire with distal occlusive balloon employs nitinol or another super-elastic material as the inner shaft or core that makes the device kink resistant;

8. the torqueable kink-resistant guidewire with distal occlusive balloon has a flexible ground tip core aligned centrally within a distally located spring coil;

9. the kink-resistant torqueable guidewire with distal occlusive balloon uses compliant, semi-compliant, or noncompliant polymer balloons, preferably such as pellathane, but in the alternative, can include other polyurethanes, Pebax, silicone, poly-isoprene, C-flex, latex, ethylene propylene, rubber and the like;

10. the torqueable kink-resistant guidewire with distal occlusive balloon is used for distal protection with Angiojet® (cross stream thrombectomy) catheters or other aspiration systems to remove the trapped particles;

11. the torqueable kink-resistant guidewire with distal occlusive balloon is used as part of an isolation system for purposes of hemolysis containment or drug infusion that may or may not include a proximal protection device;

12. the torqueable kink-resistant guidewire with distal occlusive balloon is used for embolectomy with or without an aspiration system;

13. the torqueable kink-resistant guidewire with distal occlusive balloon is useful as a centering device for other devices, such as Angiojet®; and,

14. the torqueable kink-resistant guidewire with distal occlusive balloon can be utilized to meet different profile criteria: namely,
a. a large profile such as 0.035 inch, for example, can include a coaxially aligned supporting inflation tube and a primary inflation tube arrangement to provide for mutual support and for suitable torqueing capabilities along the greater length of a torqueable kink-resistant guidewire with distal occlusive balloon; and,
b. a small profile such as 0.014 inch, for example, can include the use of a minimum cross section swaged joint connecting a proximally located inflation tube to a centrally located inflation tube of nitinol for suitable strength and for suitable torqueing capabilities along the greater length of a torqueable kink-resistant guidewire with distal occlusive balloon.

Having thus briefly described the present invention and having mentioned some significant aspects and features of the present invention, it is the principal object of the present invention to provide an occlusive guidewire system having an ergonomic handheld control mechanism prepackaged in a pressurized gaseous environment and a compatible prepackaged torqueable kink-resistant guidewire with distal occlusive balloon.

### BRIEF DESCRIPTION OF THE DRAWINGS

Other objects of the present invention and many of the attendant advantages of the present invention will be readily appreciated as the same becomes better understood by reference to the following detailed description when considered in connection with the accompanying drawings, in which like reference numerals designate like parts throughout the figures thereof and wherein:

FIG. 1 is an isometric view of an occlusive guidewire system having an ergonomic handheld control mechanism prepackaged in a pressurized gaseous environment and a compatible prepackaged torqueable kink-resistant guidewire with distal occlusive balloon, the present invention;

FIG. 2 is an exploded isometric view of the occlusive guidewire system having an ergonomic handheld control mechanism prepackaged in a pressurized gaseous environment and a compatible prepackaged torqueable kink-resistant guidewire with distal occlusive balloon;

FIG. 3 is an isometric view of the improved ergonomic handheld control mechanism and improved torqueable kink-resistant guidewire;

FIG. 4 is an exploded isometric view of the handheld control mechanism of FIG. 3;

FIG. 5 is a view of the internal mechanism assembly prior to installation between the upper housing half and the lower housing half of the handheld control mechanism;

FIG. 6 is a view of the components of FIG. 5 installed in the lower housing half;

FIG. 7 is an isometric view of the compression sealing mechanism and, in alignment, an exploded isometric view of the inflation tube sealing mechanism;

FIG. 8 is an exploded isometric view of the compression sealing mechanism;

FIG. 9 is an isometric view of the multiple guide cell seal;

FIG. 10 is a front view of the multiple guide cell seal;

FIG. 11 is a cross section view along line 11-11 of FIG. 10 showing the accommodation of several crimped crimpable inflation tubes;

FIG. 12 is a cross section view of the compression sealing mechanism along line 12-12 of FIG. 7;

FIG. 13 is a cross section view of the inflation tube sealing mechanism along line 13-13 of FIG. 7 combined with the cross section view of FIG. 12 taken along line 12-12 of FIG. 7;

FIG. 14 is a foreshortened cross section view of the torqueable kink-resistant guidewire engaged with a balloon protector prior to subsequent use of the torqueable kink-resistant guidewire with the handheld control mechanism of FIG. 1;

FIG. 15 is a foreshortened section view along line 15-15 of FIG. 14;

FIG. 16 is a cross section view along line 16-16 of FIG. 14 showing the structure of an occlusive balloon apart from the balloon protector and components associatingly connected thereto;

FIG. 17 is a plan view of a transportation coil;

FIG. 18 is a foreshortened cross section view of the torqueable kink-resistant guidewire and a balloon protector having a flared section prior to use of the torqueable kink-resistant guidewire with the handheld control mechanism of FIG. 2;

FIG. 19 is a cross section view of the torqueable kink-resistant guidewire along line 19-19 of FIG. 18;

FIG. 20 illustrates the relationship of the combined FIGS. 21a and 21b; and,

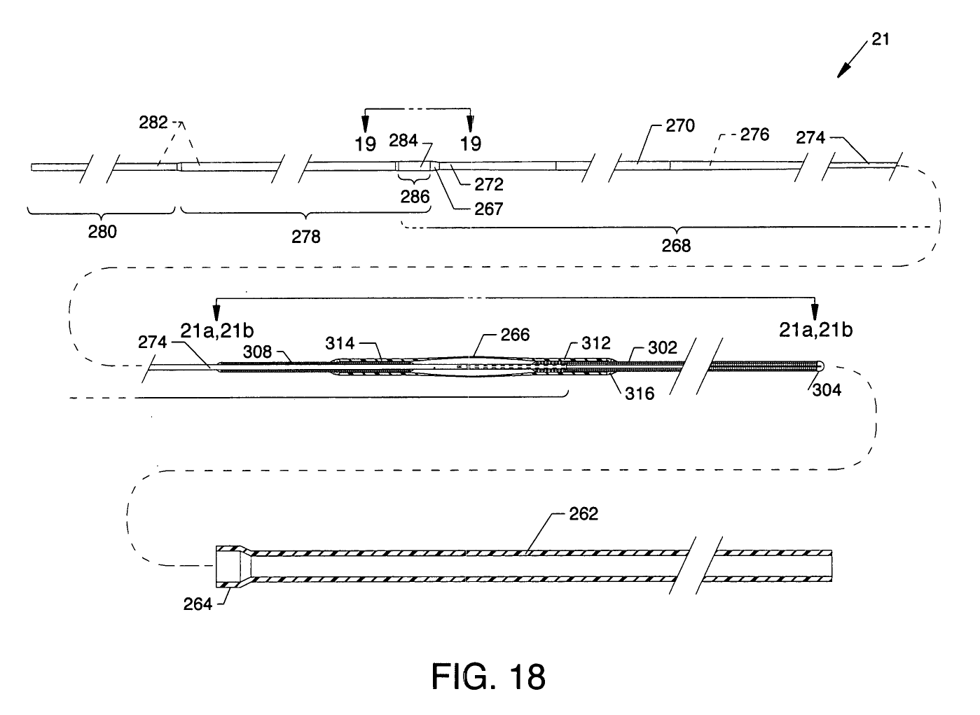

The combined FIGS. 21a and 21b are a foreshortened section view along line 21a,21b-21a,21b of FIG. 18.

### DETAILED DESCRIPTION OF THE PREFERRED EMBODIMENTS

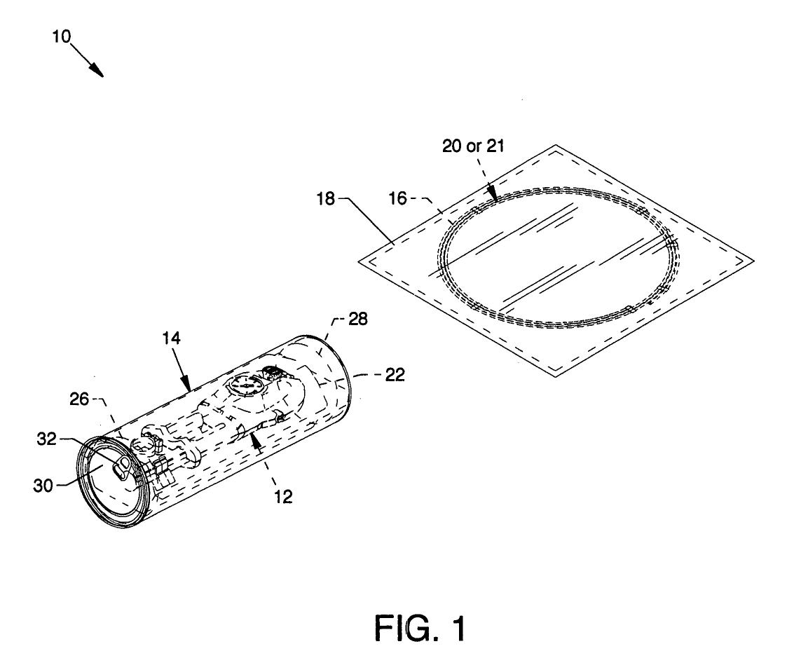

FIG. 1 is an isometric view of an occlusive guidewire system having an ergonomic handheld control mechanism prepackaged in a pressurized gaseous environment and a compatible prepackaged torqueable kink-resistant guidewire with distal occlusive balloon 10, the present invention, hereinafter, for brevity, referred to simply as an occlusive guidewire system 10. Readily visible components of the instant invention include an improved handheld control mechanism 12, previously referenced in and closely replicating the handheld control mechanism disclosed in application Ser. No. 11/217,545, sealed within a hermetically sealed container in the form of a canister 14, and a transportation coil 16 in a hermetically sealed container in the form of a pouch 18.

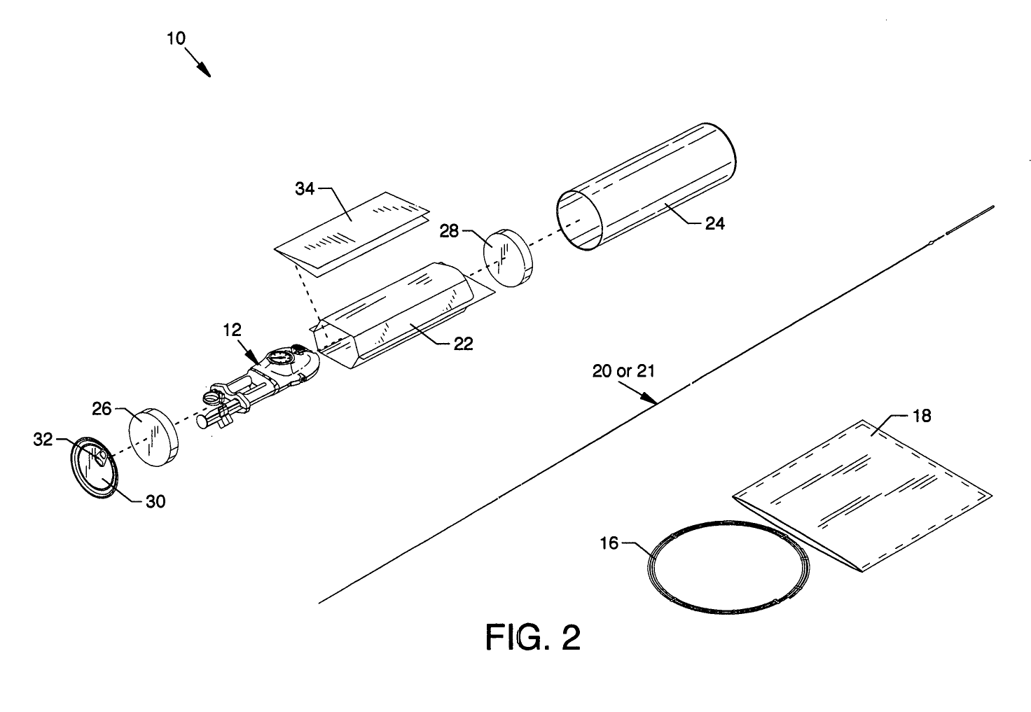

FIG. 2 is an exploded isometric view of the occlusive guidewire system having an ergonomic handheld control mechanism prepackaged in a pressurized gaseous environment and a compatible prepackaged torqueable kink-resistant guidewire with distal occlusive balloon 10, the present invention, including the handheld control mechanism 12 and one or another of a torqueable kink-resistant guidewire 20 or 21 removed from the container components. The torqueable kink-resistant guidewires 20 and 21, each of which is useable with the handheld control mechanism 12, are described later in detail. A torqueable kink-resistant guidewire 20 or 21 is sealably housed primarily within the transportation coil 16, and when sealed, both are contained within the sealed pouch 18. The torqueable kink-resistant guidewire 20 or 21 can be one of the torqueable kink-resistant guidewires, described later in detail, or can be of other suitable design. The canister 14 includes components for safekeeping and shock mounting of the handheld control mechanism 12 within the canister 14 including a spacer pouch 22 which could also be a sterile barrier or redundant sterile barrier which closely accommodates the exterior profile of the handheld control mechanism 12 and having a periphery which is closely accommodated by the interior of a canister body 24. The canister body is comprised of 20EE tin plated steel, 88 pound base weight (0.0097 nominal) and a welded side seam. The spacer pouch 22 includes a clear panel 22a and an attached Tyvek® panel 22b. Preferably the Tyvek® panel 22b can be uncoated Tyvek® 1073B with a porosity specification of 20-100 Gurly Seconds. Foam disks 26 and 28 are incorporated in the interior ends of the canister 14 for positioning of the spacer pouch 22 and the contained handheld control mechanism 12 with respect to the ends of the canister 14. A canister lid 30 having a pull ring 32 and the canister body 24 comprise the canister 14. An information packet 34 can also be included within the canister 14. The exterior of the canister 14 includes instructions, data and other useful information relating to the contents of and the use of the contents of the canister 14 as does the information packet 34. The canister lid 30 includes graphic information regarding removal of the canister lid 30 by use the pull ring 32.

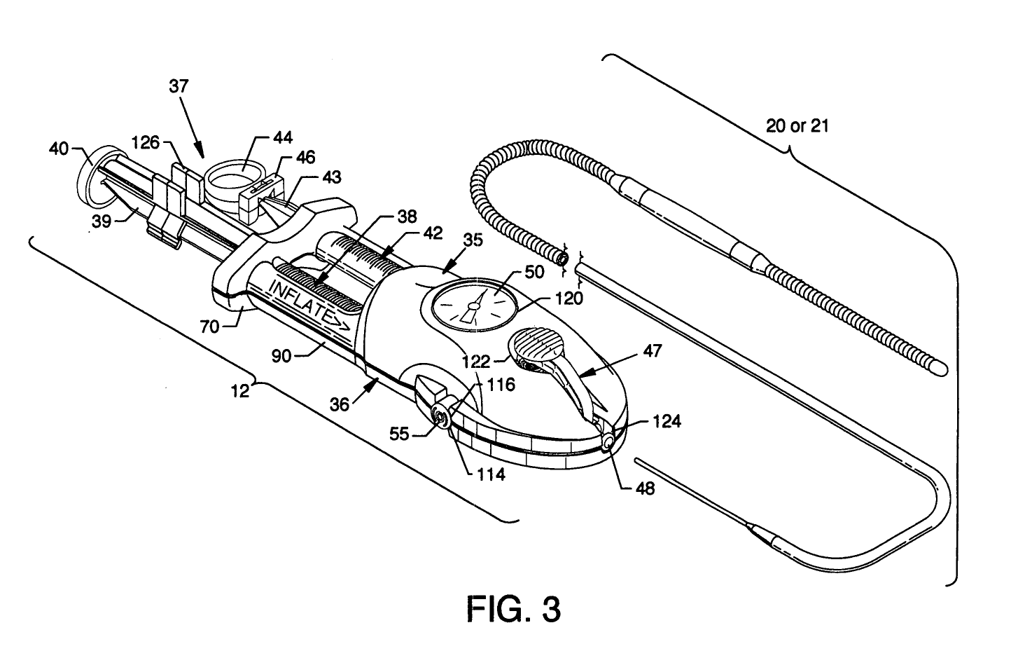

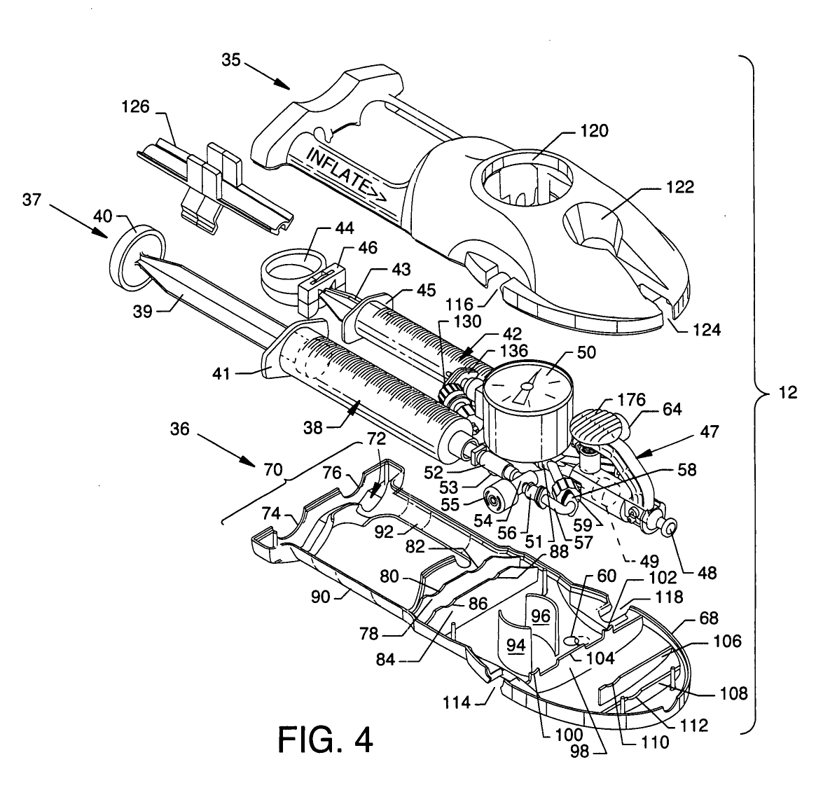

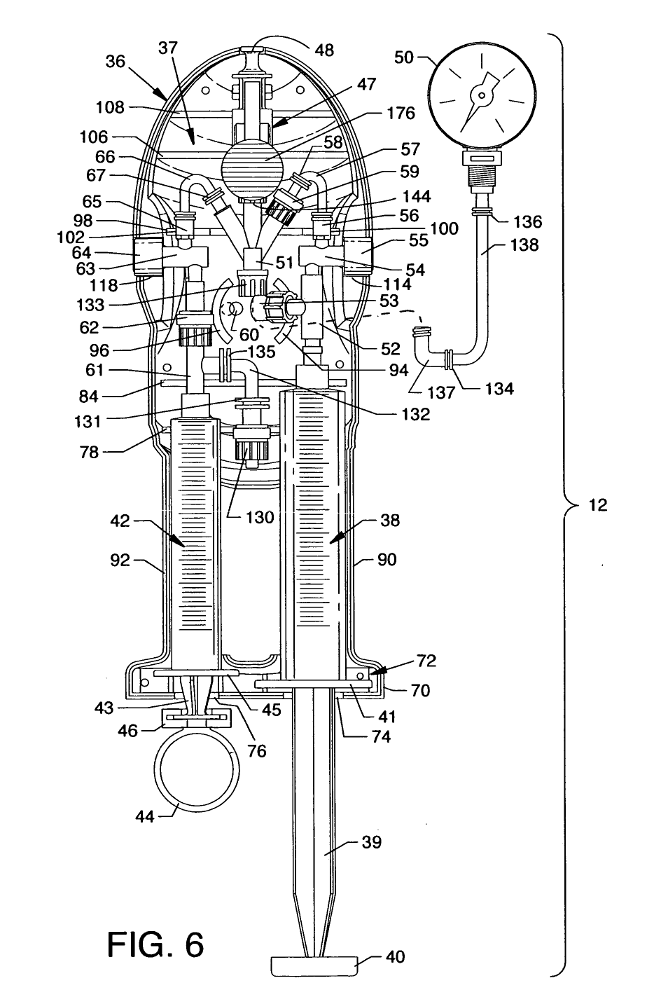

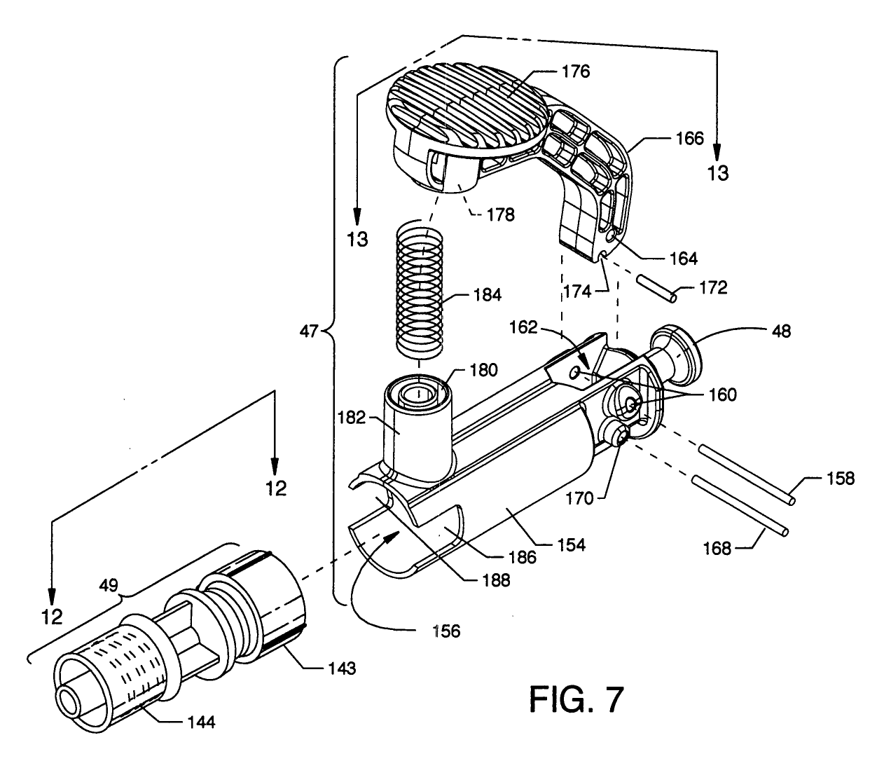

FIG. 3 is an isometric view of the improved ergonomic handheld control mechanism 12 and improved torqueable kink-resistant guidewire 20 or 21, and FIG. 4 is an exploded isometric view of the handheld control mechanism 12 of FIG. 3. With reference to FIGS. 3 and 4, the handheld control mechanism 12 is now described. The handheld control mechanism 12 is ergonomically shaped, including an ergonomically shaped upper housing half 35 and an ergonomically shaped lower housing half 36, which together encompass and serve as mounting structure for full or partial encasement of the majority of the components included in a centrally located internal mechanism assembly 37. Components of the internal mechanism assembly 37, as also shown in FIGS. 6 and 7, include a graduated inflation syringe 38 having a plunger 39, an actuator pad 40 and a flange 41; a graduated evacuation syringe 42 having a plunger 43, an actuator ring 44, a flange 45, and a plunger stop 46; an inflation tube sealing mechanism 47 including a receptor orifice 48; a compression sealing mechanism 49 (FIG. 7) intimately engaging the inflation tube sealing mechanism 47; and a pressure gauge 50, preferably calibrated in atmosphere units (ATM).

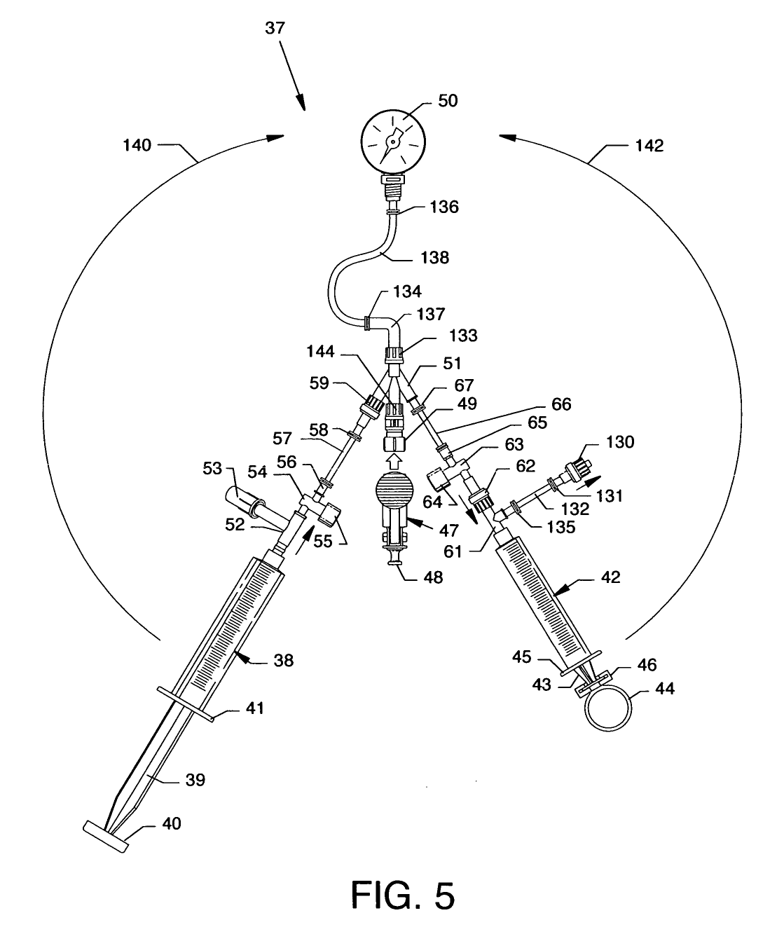

Also included, as clearly shown in FIG. 5, is the distal end of the inflation syringe 38 connecting to a four-port infusion “Y” 51 via a T-connector 52 having a low pierce force needle injection port 53 connected thereto, an inline inflation control valve 54 having an inflation control valve actuator button 55, a female slip Luer 56, a flexible plastic tube 57, a male slip Luer 58, and a check valve 59. An access hole 60, shown in FIGS. 4 and 6, extends at an angle through the lower housing half 36 in alignment with the low pierce force needle injection port 53 in order to accommodate a needle used for injection of a gas, preferably CO₂, or other inflationary substance into the inflation syringe 38.

In a closely related manner, and as shown in FIG. 5, the distal end of the evacuation syringe 42 is shown connecting to the four-port infusion “Y” 51 via a female Luer lock tee 61, a Luer connector check valve 62, an inline evacuation control valve 63 having an evacuation control valve actuator button 64, a female slip Luer 65, a flexible plastic tube 66, and a male slip Luer 67.

The inflation control valve 54 and the evacuation control valve 63 are inline valves which are normally closed to maintain a closed valve position. Depressing the inflation control valve actuator button 55 of the inflation control valve 54 or depressing the evacuation control valve actuator button 64 of the evacuation control valve 63 causes opening of the respective valve to allow passage therethrough, and releasing the inflation control valve actuator button 55 of the inflation control valve 54 or releasing the evacuation control valve actuator button 64 of the evacuation control valve 63 causes each respective valve to automatically return to the closed position.

The ergonomically shaped upper housing half 35 and ergonomically shaped lower housing half 36 encompass and serve as mounting structure for full or partial support or encasement of the majority of the components of the centrally located internal mechanism assembly 37. The mounting or containment structure of the upper housing half 35 for the most part contains corresponding and accommodating structure and functions much in the same manner as that of the lower housing half 36 for securing components of the internal mechanism assembly 37 in place against the geometry of the lower housing half 36, but is not shown for the purpose of brevity.

The lower housing half 36 is bounded by a segmented mating edge 68 and has structure for mounting of the inflation syringe 38 and the evacuation syringe 42. Such structure includes a syringe support bracket 70 characterized by a laterally oriented channel 72 having arcuate notches 74 and 76 for partially accommodating the plungers 39 and 43. The channel 72 also accommodatingly captures lower portions of the flanges 41 and 45 of the inflation syringe 38 and the evacuation syringe 42, respectively, as shown in FIG. 6. A laterally oriented syringe support bar 78 having arcuate notches 80 and 82 along the top edge, and a laterally oriented syringe support bar 84 having arcuate notches 86 and 88 along the top edge and being parallel to the syringe support bar 78, span the lower housing half 36 to offer support of the distal portions of the inflation syringe 38 and the evacuation syringe 42, respectively. Longitudinally oriented arcuate extensions 90 and 92 connect the syringe support bracket 70 to the main body of the lower housing half 36. Vertically oriented arcuate tabs 94 and 96 extend from the main body of the lower housing half 36 to assist in support of the pressure gauge 50. Another laterally oriented support bar 98 is provided that has elevated arcuate notches 100 and 102 for support of the female slip Luer 56 and the female slip Luer 65 (FIG. 5) and a center support 104 for support of the four-port infusion “Y” 51. Laterally oriented support bars 106 and 108 having arcuate notches 110 and 112, respectively, are provided for support of the inflation tube sealing mechanism 47.

Structure is also provided for accommodation of the inflation and evacuation control valve actuator buttons 55 and 64 in the form of notches about the edges of the upper housing half 35 and the lower housing half 36. In the lower housing half 36 an interrupted arcuate notch 114 is provided. The interrupted arcuate notch 114 includes a radius slightly larger than the radius of the inflation control valve actuator button 55, whereby the slightly larger radius of the interrupted arcuate notch 114 provides for guided near tangential close spaced support of the inflation control valve actuator button 55. In the upper housing half 35 a corresponding and mating interrupted arcuate notch 116 is provided to provide a function similar to that of the interrupted arcuate notch 114. Correspondingly, on the lower housing half 36 an interrupted arcuate notch 118 opposes the interrupted arcuate notch 114 and mates to another interrupted arcuate notch on the upper housing half 35 (not shown) to provide for the same function and geometry for the evacuation control valve actuator button 64. The mated combination of the interrupted arcuate notch 116 of the upper housing half 35 with the interrupted arcuate notch 114 of the lower housing half 36, as well as like structure associated with the interrupted arcuate notch 118, provides for sheltered and recessed locations for protected housing of the inflation control valve actuator button 55 and the evacuation control valve actuator button 64. The location of the inflation control valve actuator button 55 and the evacuation control valve actuator button 64 within the mated combination of the interrupted arcuate notch 116 of the upper housing half 35 with the interrupted arcuate notch 114 of the lower housing half 36, as well as like structure associated with the interrupted arcuate notch 118, requires that wanted depression of the inflation control valve actuator button 55 or the evacuation control valve actuator button 64 can only occur when needed by the operator in that the operator must make a conscious decision and dedicated effort to depress such actuator buttons. Inadvertent actuation of the inflation control valve actuator button 55 or the evacuation control valve actuator button 64 is minimized by the recessed structure surrounding the inflation control valve actuator button 55 and the evacuation control valve actuator button 64.

The upper housing half 35 includes other features not found on the lower housing half 36, including a centrally located orifice 120 in the upper region for accommodation of the pressure gauge 50, a recess 122 in the upper forward region for accommodation of some parts of the inflation tube sealing mechanism 47, and a slot 124 at the forward edge for accommodation of the portion of the inflation tube sealing mechanism 47 which has the receptor orifice 48. A configured lock 126 is provided for locking of the inflation syringe 38 to prevent inadvertent movement of the inflation syringe 38 to preclude inadvertent inflation of an inflatable balloon attached as part of the invention.

FIG. 5 is a view of the internal mechanism assembly 37 prior to installation between the upper housing half 35 and the lower housing half 36. Shown in particular in the illustration are components not previously described or shown or components previously partially shown, now shown for completeness, including the evacuation control valve 63 and the evacuation control valve actuator button 64, Luer connector check valve 62 connected to the evacuation control valve 63, a female Luer lock tee 61 connecting Luer connector check valve 62 to the distal end of the evacuation syringe 42, and a Luer connector purge check valve 130 connected by a flexible plastic tube 132 and male slip Luers 131 and 135 to the central portion of the female Luer lock tee 61. Also shown at the distal end of the four-port infusion “Y” 51 is a Luer connector 133 of the four-port infusion “Y” 51 connected to an angled connector 137, a male slip Luer 134, and a flexible plastic tube 138 which is, in turn, attached to the pressure gauge 50 by a male slip Luer 136. Arrows 140 and 142 indicate the direction of reorientation of the components proximal to the flexible plastic tubes 57 and 66 about the flexible plastic tubes 57 and 66 when installed between the upper housing half 35 and the lower housing half 36, such as shown in FIGS. 3 and 6.

FIG. 6 is a view of the components of FIG. 5 installed in the lower housing half 36. The handheld control mechanism 12 is conveniently packaged in the canister 14 ready for use by a physician. The packaging process is accomplished in several steps. The handheld control mechanism 12 is first backfilled with CO₂ by purging a gas supply line and then introducing CO₂ therethrough to fill the inflation syringe 38 with CO₂, and subsequently tested, such as by the use of a MAPtest 3050 Packaging Analyser, such as by Hitech Instruments Ltd. of Luton, England. Backfilling is done by depressing the inflation control valve actuator button 55 and the evacuation control valve actuator button 64 to open the inflation control valve 54 and the evacuation control valve 63, respectively, and then by depressing the actuator pad 40 of the inflation syringe 38 to position plunger 39 of the inflation syringe 38 to the fully depressed position. Then, withdraw and depress the actuator ring 44 through several cycles. Release the evacuation control valve actuator button 64. Positioned as just described, pressurized CO₂ is introduced by a needle at the distal end of the purged gas line by aligning such a needle through the access hole 60 at the lower housing half 36 and then piercing the low pierce force injection port 53 and then introducing CO₂ directly to the T-connector 52 and thence to the inflation syringe 38 causing the plunger 39 to reposition outwardly and also through the open inflation control valve 54, the four-port infusion “Y” to the evacuation control valve 63 via the closely associated connecting plastic tubes 57 and 66 and other associated devices. The evacuation control valve 63 is closed and the actuator pad 40 connected to the inflation syringe plunger 39 is depressed flushing the entire system with CO₂. After repeating the filling cycle several times, the needle is removed from the low pierce force injection port 53 and the inflation control valve actuator button 55, thereby closing the inflation control valve 54 and the evacuation control valve 63, thereby trapping and containing CO₂ within the handheld control mechanism 12, as just described. CO₂ quality is then tested, such as by the use of a MAPtest 3050 Packaging Analyser, where a hypotube is made to penetrate through the receptor orifice 48 and then the compression sealing mechanism 49 to sample the quality of the CO₂ residing on the handheld control mechanism 12. Upon a successful quality check, repurging and refilling can be accomplished. A suitable desirable amount of CO₂ can be expressed as 98 percent. Preparation is also made to prepare for introduction of the CO₂ laden handheld mechanism 12 into the canister body 24 and for pressurization thereof. First, a foam disk 28 is inserted into the opening of the canister body 24 and the lock 126 is installed on the shaft of the plunger 43. The CO₂ laden handheld mechanism 12 is inserted into the open end of the spacer pouch 22 and the open end is sealed, then the spacer pouch 22 is appropriately folded at the edge boundaries and inserted, along with the information packet 34 and foam disk 26, into the canister body 24 and then placed into a sealed chamber which includes a mechanism for installing the canister lid 30 and attached pull ring 32, and which also includes a vacuum source and a pressurized gas source. The sealed chamber is then flushed multiple times and vacuum applied and vacuum drawn a number of times to fill the sealed chamber, thereby causing the CO₂ to permeate the spacer pouch 22 through the Tyvek® panel 22b to surround the handheld control mechanism 12 with CO₂. Then, application and sealing of the canister lid 30 and attached pull ring 32 to the canister body 24 is accomplished. A package leak detector is used to detect any leaks in the canister 14. Control tests can also be applied to control canisters 14 to sample proper CO₂ levels. The sealed canister 14 is then processed, such as by Steris Isomedix of Chicago Ill., where the sealed canister 14 is placed into the influence of cobalt 60 long enough to receive 25-42 KiloGray (kGy) of gamma radiation. A partial pressure minimum of 98% CO₂ is held at the time of manufacture and a minimum of 95% CO₂ is held after two years.

FIG. 7 is an isometric view of the compression sealing mechanism 49 and, in alignment, an exploded isometric view of the inflation tube sealing mechanism 47. The compression sealing mechanism 49 and the inflation tube sealing mechanism 47 are closely related to patent application Ser. No. 10/838,464 entitled “Gas Inflation/Evacuation System and Sealing System Incorporating a Compression Sealing Mechanism for Guidewire Assembly Having Occlusive Device” filed on May 04, 2004, which is hereby incorporated herein in its entirety by reference, and application Ser. No. 10/838,468 entitled “Guidewire Assembly Including a Repeatably Inflatable Occlusive Balloon on a Guidewire Ensheathed with a Spiral Coil” filed on May 04, 2004, which is hereby incorporated herein in its entirety by reference.

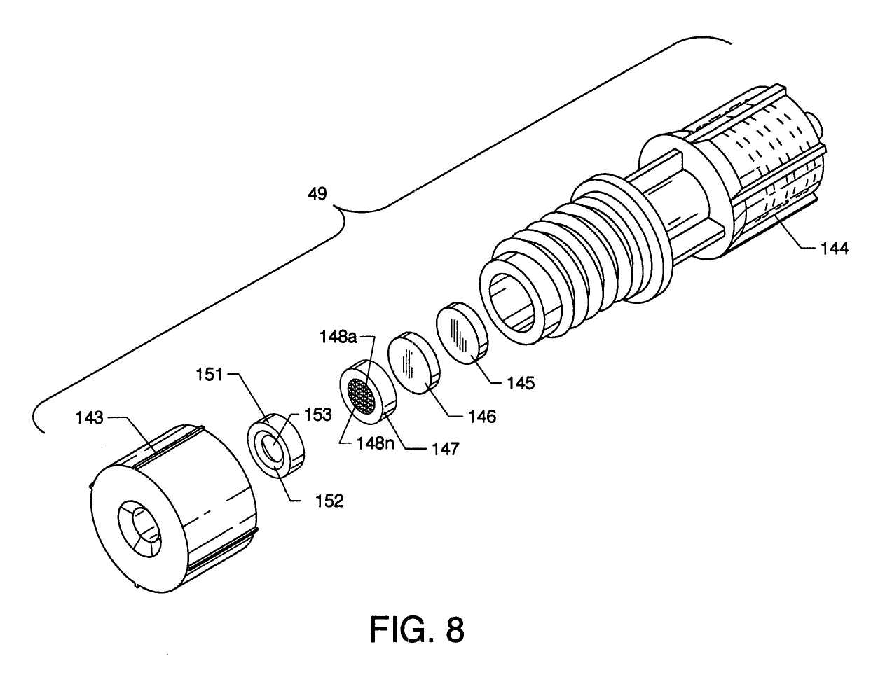

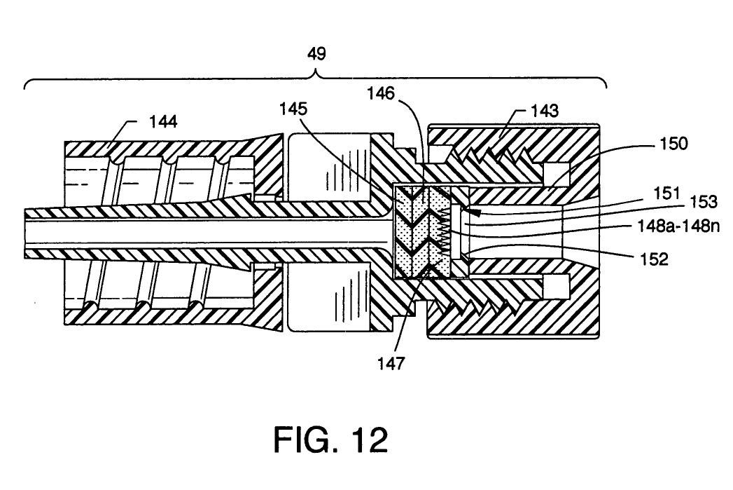

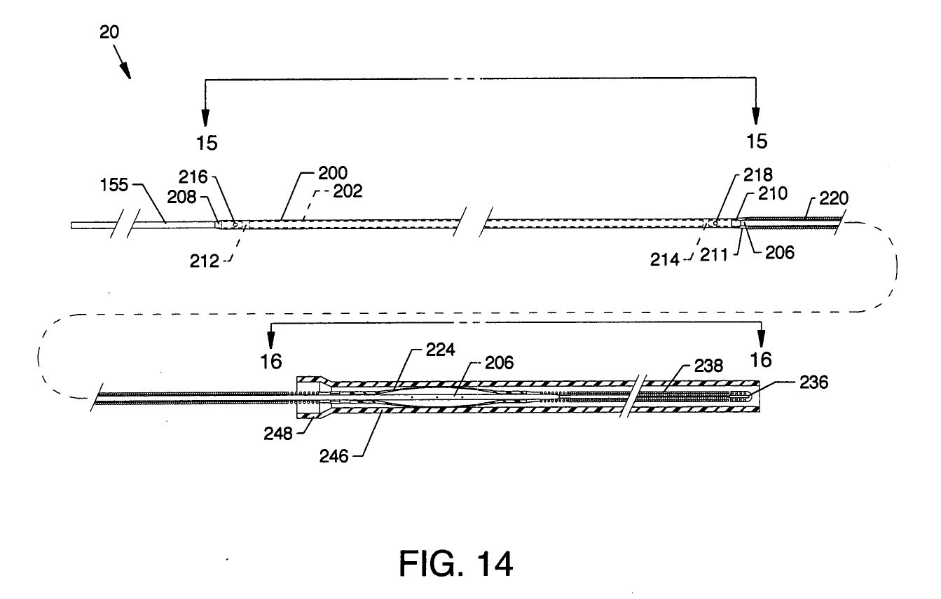

FIG. 8 is an exploded isometric view of the compression sealing mechanism 49 incorporating additional components, as well as a preponderance of the components and teachings in patent application Ser. No. 10/930,528 entitled “Low Pierce Force Needle Port” filed on Aug. 31, 2004, which is hereby incorporated herein in its entirety by reference. Major visible components, as well as other components of the compression sealing mechanism 49, include a sealing cap 143 and a male Luer connector 144. Interior components of the compression sealing mechanism 49 include puncturable self-sealing seals 145 and 146 and a puncturable self-sealing multiple guide cell seal 147, each seal being of resilient construction and each accommodatingly sealing against the outer surface of a crimpable inflation tube, such as, but not limited to, a crimpable inflation tube 155 at the proximal end of the torqueable kink-resistant guidewire 20, shown generally in FIG. 1, and shown more specifically in FIG. 14, or a crimpable inflation tube 280 at the proximal end of the torqueable kink-resistant guidewire 21, shown in FIG. 18. The puncturable self-sealing seals 145 and 146 and the puncturable self-sealing multiple guide cell seal 147 are redundant very low durometer seals in intimate sequential contact with one another or spaced away from each other to effect a multiple effort unified seal around a crimpable inflation tube such as exemplified by the crimpable inflation tube 155 at the proximal end of the torqueable kink-resistant guidewire 20 after sterilization and when pierced by one or more of the multiple crimpable inflation tubes 155. A deflection ring 151 includes an internal annular ramp 152 having a passage 153 extending along the centerline and the length of the deflection ring 151. The deflection ring 151 aligns within the male Luer connector 144 to interface between the tubular extension 150 (FIG. 12) of the sealing cap 143 and the multiple guide cell seal 147.

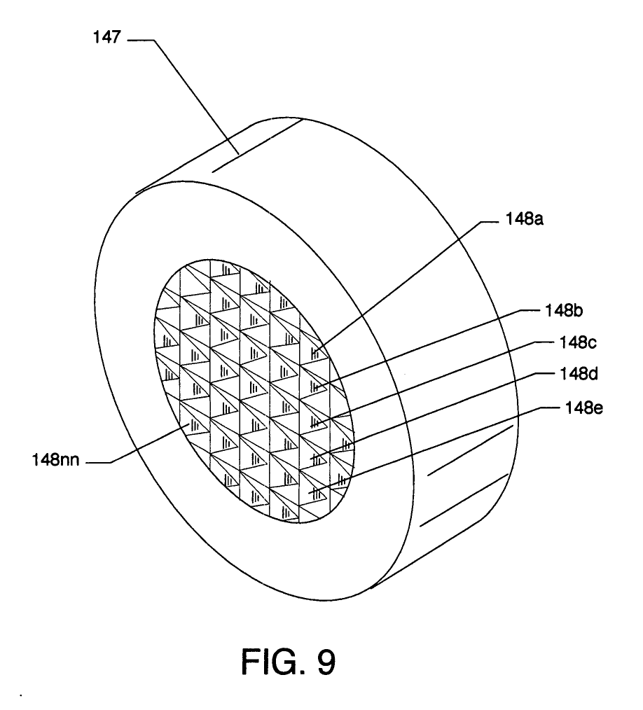

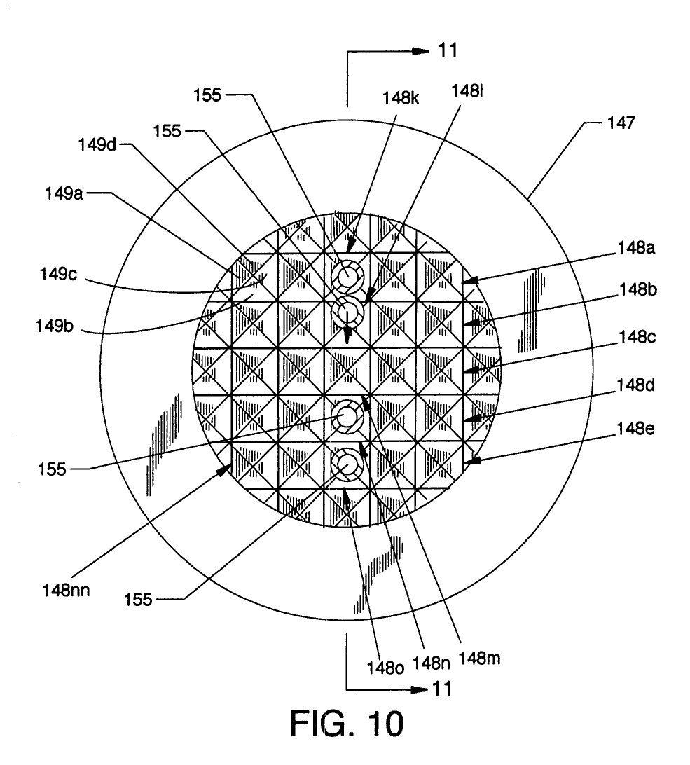

FIG. 9 is an isometric view of the multiple guide cell seal 147, and FIG. 10 is a front view of the multiple guide cell seal 147 including proximally located surface guide cells 148a-148nn where each figure also illustrates the pyramidal shape of the guide cells 148a-148nn. For the purpose of brevity and clarity, numbered identification tags are included for the centrally located guide cells 148a-148nn which are for the most fully configured. Adjoining partial structures closely resembling the guide cells 148a-148nn are shown, but not enumerated. The guide cells 148a-148nn are indented into the proximal surface of the multiple guide cell seal 147 to direct or steer multiple crimpable inflation tubes 155 one at a time away from each other, thereby reducing the probability of an additional crimpable inflation tube 155 sliding directly along another previously placed crimpable inflation tube 155 and tearing or enlarging one or more of the silicone seals 145, 146 and the multiple guide cell seal 147, thereby increasing the ability to obtain a favorable puncturing outcome whereby separate puncture hole sets for each of the crimpable inflation tubes 155 are formed being spaced across the seals. Although the guide cells 148a-148nn are shown incorporating pyramidal shapes, other suitable guide cell shapes, such as, but not limited to, conical, square, hemispherical and the like may also be incorporated within the scope of the invention.

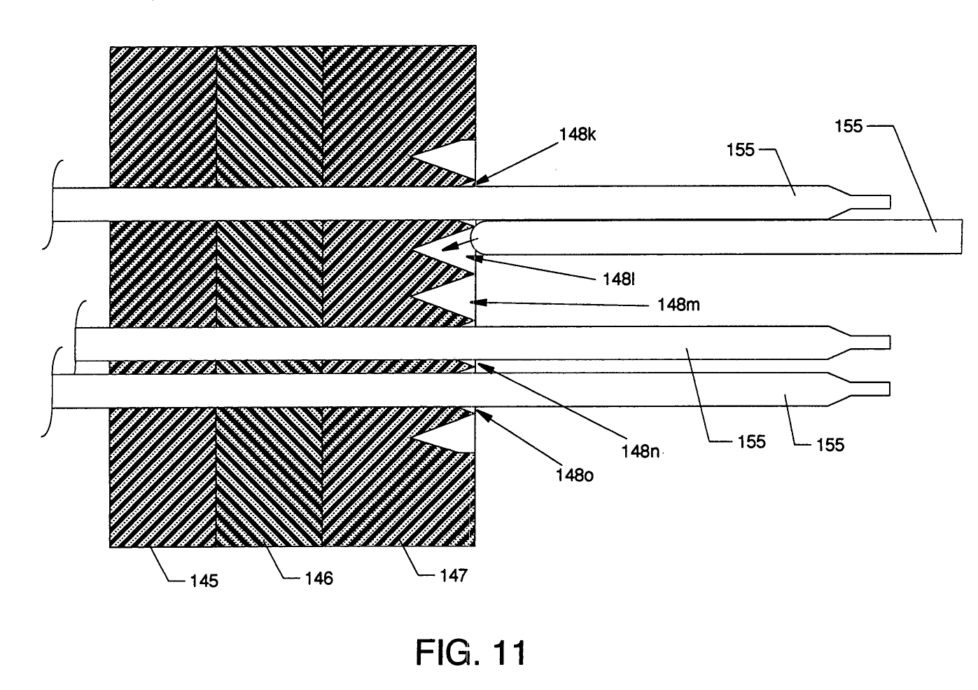

FIG. 11 is a cross section view along line 11-11 of FIG. 10 showing the accommodation of several crimped crimpable inflation tubes 155 first by the multiple guide cell seal 147 and thence by the seals 146 and 145. The pyramidal shape of the indentated guide cells 148a-148nn is formed by a plurality of adjacent intersecting steep sloping sidewalls 149a-149d, shown in FIG. 10, culminating in an apex to comprise the structure of each of the guide cells 148a-148nn. The steep sloping sidewalls 149a-149d (FIG. 10) comprising each of the guide cells 148a-148nn form the slightly larger structure of the guide cells 148a-148nn with respect to the size of a crimpable inflation tube 155 so that: (1) two of the crimpable inflation tubes 155 cannot occupy the same guide cell 148a-148nn, such as shown by the two crimpable inflation tubes 155 at guide cells 148n and 148o, respectively, and (2) a crimpable inflation tube 155 hitting or abutting an in-place crimpable inflation tube, such as crimpable inflation tube 155 at guide cell 148k, will physically be denied access to the guide cell 148k, but will be deflected by one of the adjoining steep sloping sidewalls 149a-149d of an adjacent guide cell, such as guide cell 148l, to be guided into such adjacent guide cell which is most under the center of the crimpable inflation tube 155. Another and third aspect, as shown in FIG. 12, is the relationship of the deflection ring 151 to the seals 145 and 146 and to the guide cells 148a-148nn of the multiple guide cell seal 147. The annular ramp 152 of the deflection ring 151 is instrumental in ensuring that the crimpable inflation tubes 155 are guided away from the edge of the multiple guide cell seal 147 and into the guide cells 148a-148nn thereof for proper sealing therewith, as previously described, and that the crimpable inflation tubes 155 can be deflectingly located away from the region of the compressed and more difficult to penetrate peripheral boundary material of the seals that are compressed by the tubular extension 150. In the alternative, the compression of the peripheral boundary material of the seals can be minimized by providing a thinner wall version of the deflection ring 151 to space the compression area of the seals further from the guide cells 148a-148nn, thereby resulting in a minimum surface sealing face whereby distortion of the structure of the guide cells 148a-148nn is minimized or nonexistent. Accordingly, the material adjoining the periphery of the multiple guide cell seal 147 and the guide cells 148a-148nn provides additional elastic material between the clamped peripheral seal section and the point of seal penetration so that the material of the seals is less likely to tear, thus minimizing the potential of leakage.

FIG. 12 is a cross section view of the previously described compression sealing mechanism 49 along line 12-12 of FIG. 7. Shown in particular is the relationship of the deflection ring 151 to the seals 145 and 146 and to the guide cells 148a-148nn of the multiple guide cell seal 147.

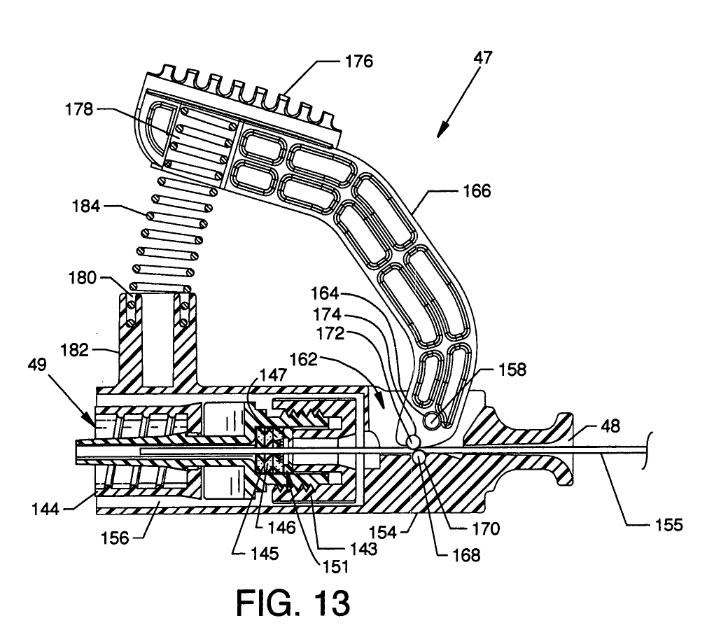

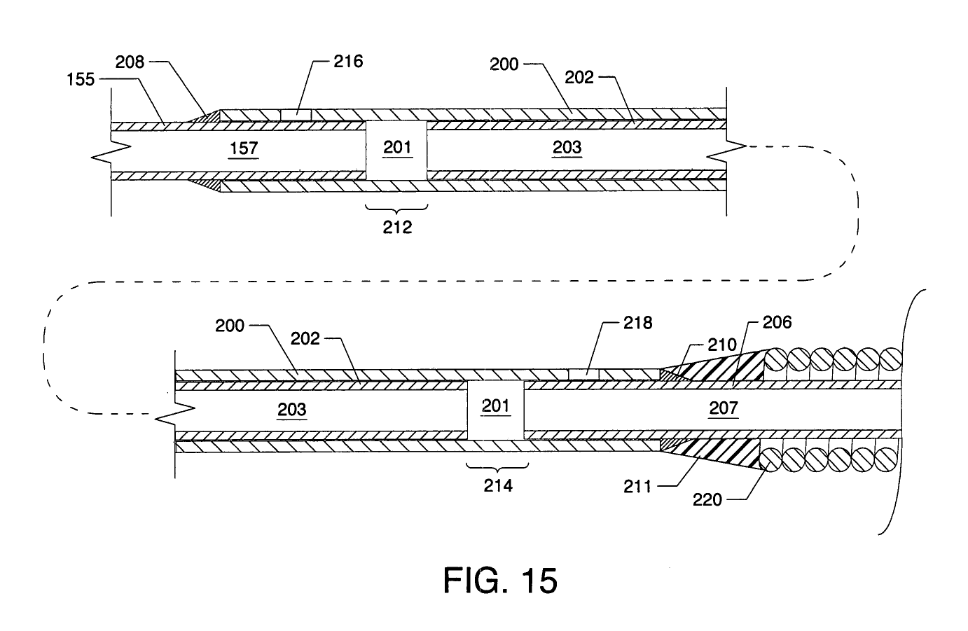

FIG. 13 is a cross section view of the inflation tube sealing mechanism 47 along line 13-13 of FIG. 7 combined with the cross section view of FIG. 12 taken along line 12-12 of FIG. 7. The invention is further described with further understood reference to FIG. 7 and other previously described figures. The inflation tube sealing mechanism 47 includes a configured body 154 of generally tubular shape and including a passageway 156 for mated accommodation of the sealing cap 143 of the compression sealing mechanism 49 therein. Also included is a pivot dowel pin 158, preferably of hardened steel, which aligns through a hole set 160 in the body 154 and through a cavity 162 in the body 154. The cavity extends along and across one end of the body 154 for accommodation of the lower end of a geometrically configured pivotable handle 166, as well as for accommodation of the pivot dowel pin 158, which extends through a horizontally oriented pivot hole 164 located in the lower region of the pivotable handle 166. A stationary pincer dowel pin 168, preferably of hardened steel, aligns in a transversely oriented hole 170, the central part of which is truncated, the truncated central part being located at the bottom of the cavity 162. The upper region of the stationary pincer dowel pin 168 protrudes slightly above the lower surface of the cavity 162, as shown in FIG. 13, in order to accommodate a surface of a crimpable inflation tube 155. An actuatable pincer dowel pin 172, preferably of hardened steel, aligns and affixes within a truncated hole 174 at the lower region of the handle 166 and protrudes slightly below the lower surface of the lower region of the handle 166. An actuator pad 176, preferably having a tactile surface, is located at the upper end of the handle 166 in close proximity to a spring receptor cavity 178. Another spring receptor cavity 180, which is annular in shape, is located in a cylindrical post 182 extending in vertical orientation from the end of the body 154. Opposing ends of a return spring 184 mount in and between the spring receptor cavity 178 and the spring cavity 180 to position the handle 166 in an open position with respect to the actuatable pincer dowel pin 172 and the stationary pincer dowel pin 168 for accommodation of the crimpable inflation tube 155. Horizontally opposed notches 186 and 188 are located in one end of the body 154 to accommodate other structure of the internal mechanism assembly 37, such as structure of the four-port infusion “Y” 51. The handle 166 is operated about the pivot dowel pin 158 to forcefully urge the actuatable pincer dowel pin 172 with sufficient force against the crimpable inflation tube 155 and the underlying stationary pincer dowel pin 168 to simultaneously seal and sever the crimpable inflation tube 155. Such simultaneous sealing and severing of the crimpable inflation tube 155 results in forcible pressure applied at opposite locations of the crimpable inflation tube 155 to reshape the crimpable inflation tube 155, whereby a lumen 157 (FIG. 15) of the crimpable inflation tube 155 is sealed by the inwardly reshaped crimpable inflation tube 155. Such action maintains pressure in the sealed and severed distal portion of the crimpable inflation tube 155, thereby maintaining inflation of an occlusive balloon 224, as later described in detail. The same principles of use and design of the inflation sealing tube mechanism 47 and the compression sealing mechanism 49 apply to a similar feature crimpable inflation tube 280 at the proximal end of the torqueable kink-resistant guidewire 21 shown in FIG. 18.

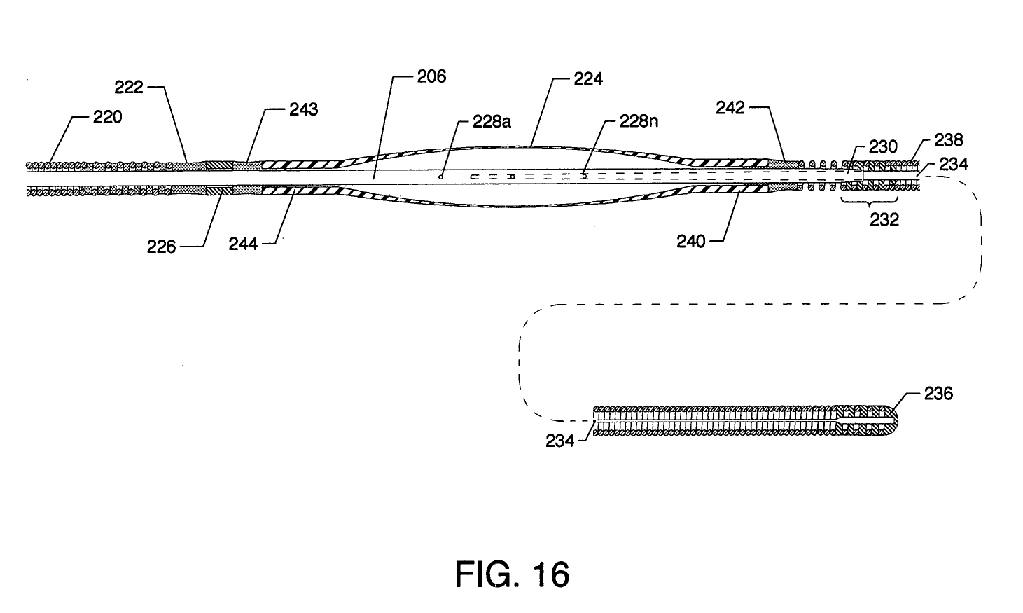

FIG. 14 is a foreshortened cross section view of the torqueable kink-resistant guidewire 20 engaged with a balloon protector 246 prior to subsequent use of the torqueable kink-resistant guidewire 20 with the handheld control mechanism 12 of FIG. 1 or other such devices having inflation tube crimping capabilities. FIG. 15 is a foreshortened section view along line 15-15 of FIG. 14 showing the general structure of coaxially arranged and aligned torqueable and flexible supporting inflation tube 200 and primary inflation tube 202 and components associatingly connected thereto. FIG. 16 is a cross section view along line 16-16 of FIG. 14 showing the structure of an occlusive balloon 224 and components associatingly connected thereto. With reference to FIGS. 14, 15 and 16, the torqueable kink-resistant guidewire 20 is now described. The supporting inflation tube 200 and primary inflation tube 202 extend in coaxial alignment along the greater length of the torqueable kink-resistant guidewire 20 between the crimpable inflation tube 155 and an inflation tube 206 which is also torqueable and flexible. The supporting inflation tube 200 and primary inflation tube 202 include lumens 201 and 203, respectively. The primary inflation tube 202 extends generally, but not entirely, along the inner length of the lumen 201 of the supporting inflation tube 200. Although the supporting inflation tube 200 and primary inflation tube 202, which are in mutual coaxial alignment, align between the crimpable inflation tube 155 and an inflation tube 206, there is only a direct solder connection 208 at, between and extending along the junction formed by the inner diameter of the proximal end of the supporting inflation tube 200 and the outer diameter of the distal end of the crimpable inflation tube 155 which aligns within the proximal end of the supporting inflation tube 200, and only a direct solder connection 210 at, between and extending along the junction formed by the inner diameter of the distal end of the supporting inflation tube 200 and the outer diameter at the proximal end of the inflation tube 206 which aligns within the distal end of the supporting inflation tube 200. A UV urethane adhesive strain relief adheres to and extends between the direct solder connection 210 and the one end of the proximally located spring coil 208, and along a short portion of the inflation tube 206. The primary inflation tube 202 aligns closely and freely and without any solder connection along and within the major length of the inner diameter of the supporting inflation tube 200. A short space 212 exists between the distal end of the crimpable inflation tube 155 and the proximal end of the primary inflation tube 202, and a short space 214 exists between the distal end of the primary inflation tube 202 and the proximal end of the inflation tube 206. The coaxial relationship of the primary inflation tube 202 to the supporting inflation tube 200 is that of mutual and nonbinding flexible multiple wall support, whereby during angular and free-floating flexing, the short spaces 212 and 214 allow for nonrestrictive displacement of the primary inflation tube 202 within and along the supporting inflation tube 200, as required. The supporting inflation tube 200 in combination with the underlying primary inflation tube 202 provides greatly enhanced stiffness. The primary inflation tube 202 is left free-floating to enhance stiffness of the combined supporting inflation tube 200 and primary inflation tube 202 while keeping the possibility of buckling of the combined unit to a minimum. An inspection hole 216 is included at the proximal end of the supporting inflation tube 200 for visual inspection of the closely associated solder connection 208 and an inspection hole 218 is included at the distal end of the supporting inflation tube 200 for visual inspection of the closely associated solder connection 210. A proximally located spring coil 220, preferably of stainless steel, is bonded by adhesive 222 in place on the inflation tube 206 immediately proximal to the occlusive balloon 224, extending a distance equal to the effective working length of the torqueable kink-resistant guidewire 20. For purposes of illustration, the proximally located spring coil 220 extends a distance equal to, but not limited to, approximately 4 feet. The proximally located spring coil 220 is wound before assembly whereby the consecutive coils are impinging upon the previous coil, making the proximally located spring coil 220 very tight and stiff. The inner diameter of the proximally located spring coil 220 tightly approximates the outer diameter of the underlying inflation tube 206. The function of the proximally located spring coil 220 is twofold. First, the stiffness of the proximally located spring coil 220 adds stiffness to the somewhat flexible inflation tube 206. Secondarily, the proximally located spring coil 220 provides kink resistance, since the inflation tube 206 has a propensity for kinking therealong. A radiopaque marker band 226 is also secured over and about the inflation tube 206 just distal of the adhesive 222. The distal portion of the inflation tube 206 includes a plurality of inflation orifices 228a-228n each communicating with the centrally located lumen 207 of the inflation tube 206 and also includes a tapered tip 230 which accommodates and is connected by a solder connection 232 to a flexible ground tip core 234. The proximal portion of the flexible ground tip core 234 is round and the distal portion is flat ground for flexibility enhancement, as best shown in the lower portion of FIG. 16. The flexible ground tip core 234 extends proximally from the point of entry into the tapered tip 230 of the inflation tube 206 to the region of the inflation orifices 228a-228n, and extends distally from the point of entry into the tapered tip 230 to terminate at a rounded distal tip 236. The proximal end of a distally located spring coil 238 having qualities similar to the proximally located spring coil 220 is attached to the exterior of the tapered tip 230 of the inflation tube 206 by the solder connection 232. To enable the inner diameter of the distally located spring coil 238 to press onto the distal end of the inflation tube 206 and for a better bond, the distal end of the inflation tube 206 utilizes the taper of the tapered tip 230, which also imparts a strain relief from the inflation tube 206 to the much more flexible ground tip core 234. The distally located spring coil 238 extends from the tapered tip 230 over, about and along the flexible ground tip core 234 to terminate at the rounded distal tip 236, which is a rounded solder structure. Communication along the torqueable kink-resistant guidewire 20 is maintained along and between the lumen 157 of the crimpable inflation tube 155, a short distance along the lumen 201 of the supporting inflation tube 200 at the short space 212, the lumen 203 of the primary inflation tube 202, a short distance along the lumen 201 of the supporting inflation tube 200 at the short space 214, the lumen 207 of the inflation tube 206, and the plurality of inflation orifices 228a-228n at the distal end of the inflation tube 206.

The occlusive balloon 224 is secured over and about the distal portion of the inflation tube 206 utilizing a unique process in order to facilitate a minimum diameter profile, such as 0.035 inch, for purpose of example and illustration. Preferably, a compliant balloon material comprising the occlusive balloon 224 could be Pellethane 2363 80AE or other suitable material. The “80A” refers to the durometer, or softness, of the polymer and the “E” refers to the extrusion grade. The extrusion grade of the 80A (80AE) material contains additional components that enable extrudability or possibly elongation. By experimentation, this grade (80AE) has proven to out perform the standard 80A in the ability to expand to a greater extent. The method of balloon bonding is first to bond the distal balloon neck 240 to the exterior of the inflation tube 206 using an adhesive 242, which preferably is a UV cure urethane adhesive, ensuring that adhesive 242 is located between the inner bore of the distal balloon neck 240 and the exterior of the inflation tube 206, as well as also extending from the edge of the distal balloon neck 240 a short distance along the inflation tube 206. Then, a short length (1.0″ length of 0.038″×0.065″ for purposes of illustration and example) of silicone tube fractionally larger than the occlusive balloon 224 and distal balloon neck 240 is slid over and about the cured adhesive 242 and the distal balloon neck 240 at the distal end of the occlusive balloon 224. The proximal balloon neck 244 is pulled gently in a proximal direction as the silicone tube is slid in light frictional engagement in a proximal direction over and about the occlusive balloon 224, thereby stretching the occlusive balloon 224 proximally. Such stretching is continued until the proximal edge of the silicone tube is fully engaged over and about the body of the occlusive balloon 224. This configuration compressingly holds the balloon body down and in the extended and stretched position while the proximal balloon neck 244 is bonded using an adhesive 243 similar to adhesive 242 ensuring that adhesive 243 is located between the inner bore of the proximal balloon neck 244 and the exterior of the inflation tube 206, as well as also extending from the edge of the proximal balloon neck 244 a short distance along the inflation tube 206 to meet the radiopaque marker band 226. The silicone tube is subsequently removed. A suitably sized balloon protector 246 having a flared section 248, shown in FIG. 14, with an ID of 0.034″, for example, is slid over the occlusive balloon 224 while rotating the balloon protector 246 so the folds of the occlusive balloon 224 are turned and rolled. When the device is hydrophilically coated, the cure temperature of the coating exposes the entire device to 145° F. which “sets” the occlusive balloon 224 in this wrapped condition so that when the physician uses the device, the profile is well under 0.038 inch, for example.

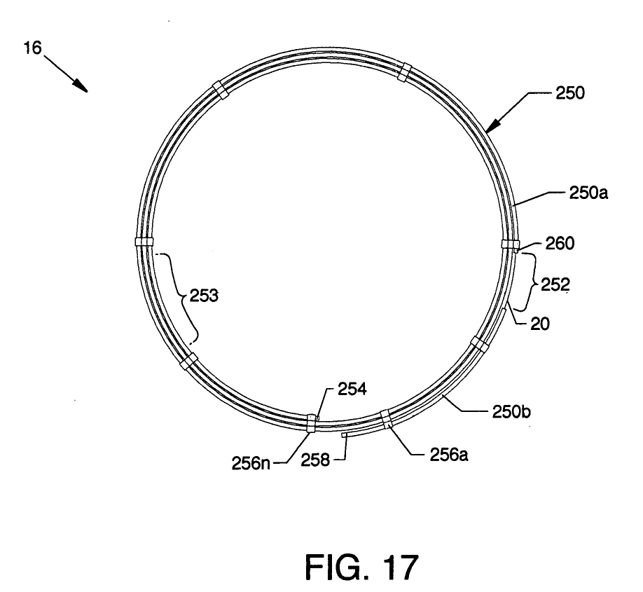

FIG. 17 is a plan view of the transportation coil 16 which protectively accommodates the length of the torqueable kink-resistant guidewire 20, as well as the balloon protector 246. A coiled segmented flexible tube 250 having an inner diameter capable of accommodating the width of the flared section 248 of the balloon protector 246 and a sufficient length to accommodate the length of the combined torqueable kink-resistant guidewire 20 and balloon protector 246 is arranged in spiral fashion. The segmented flexible tube 250 includes an elongated flexible tube segment 250a and a short flexible tube segment 250b, there being a gap 252 separating the elongated flexible tube segment 250a and the short flexible tube segment 250b. Preferably, the inner end 254 of the elongated flexible tube segment 250a is closed. A plurality of clips 256a-256n fasten adjacent coils of the segmented flexible tube 250 to maintain an orderly structure. In a storage operation, the proximal end of the crimpable inflation tube 155 of the torqueable kink-resistant guidewire 20 is inserted first into an end 258 of the short flexible tube segment 250b, thence along the gap 252, and then into an end 260 of the elongated flexible tube segment 250a and urged along and through the length of the elongated flexible tube segment 250a, thereby training the other components of the torqueable kink-resistant guidewire 20 and an attached balloon protector 246 within the structure of the segmented flexible tube 250. Removal of the torqueable kink-resistant guidewire 20 and attached balloon protector 246 from the segmented flexible tube 250 of the transportation coil 16 is accomplished by grasping the torqueable kink-resistant guidewire 20, which is exposed in the gap 252, followed by urging outwardly from the coiled segmented flexible tube 250. In the alternative, another gap, designated at 253, can be included prior to and spaced from the end 254 for additional access to any of the torqueable kink-resistant guidewires 20 or 21.

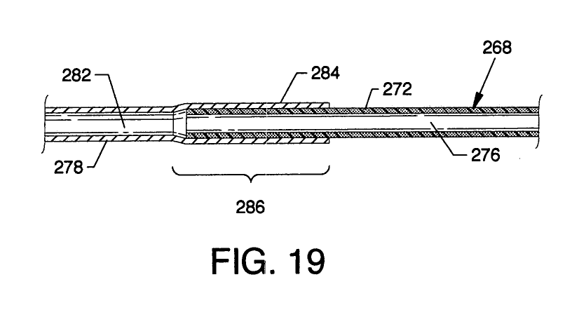

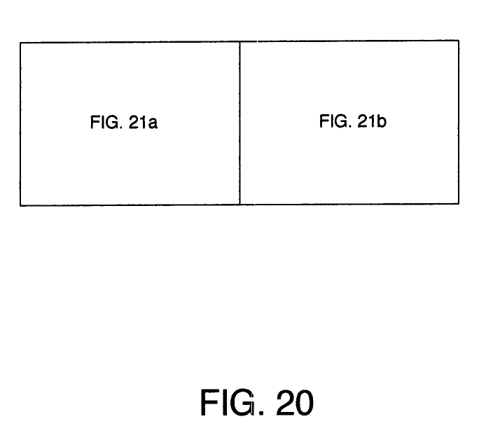

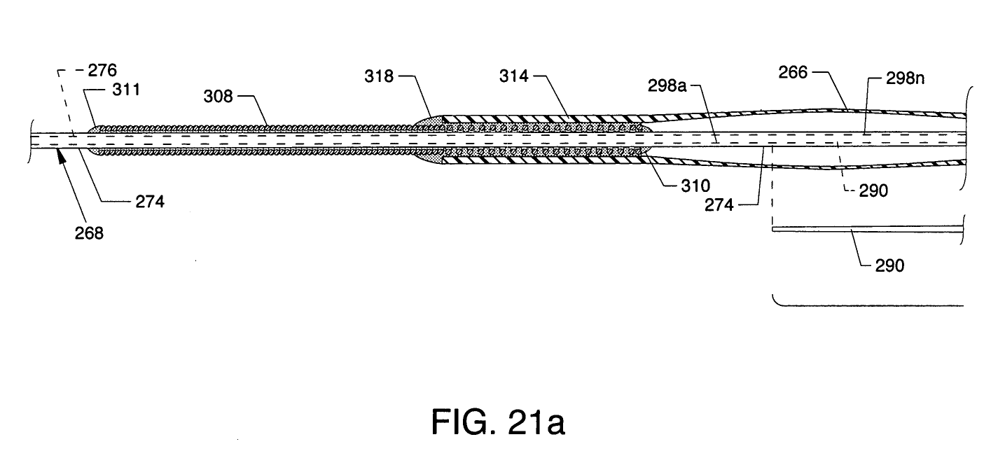

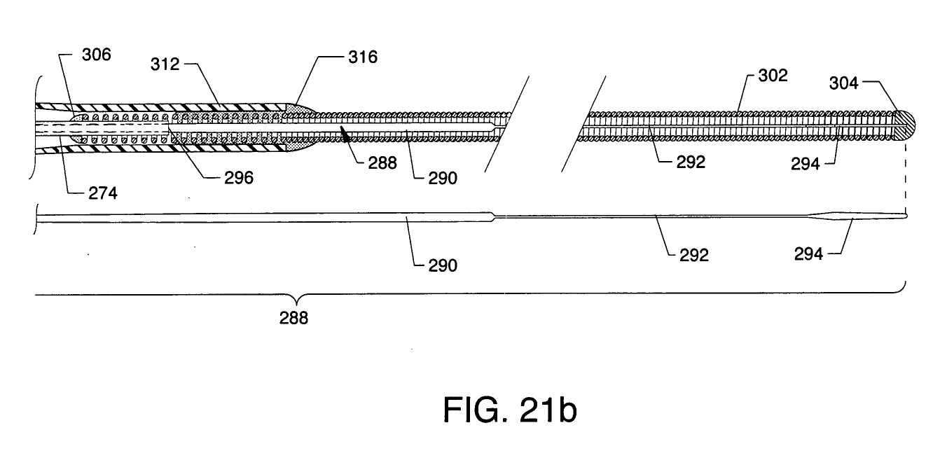

FIG. 18 is a foreshortened cross section view of the torqueable kink-resistant guidewire 21 and a balloon protector 262 having a flared section 264 prior to subsequent use of the torqueable kink-resistant guidewire 21 with the handheld control mechanism 12 of FIG. 1 or other such devices having inflation tube crimping capabilities. FIG. 19 is a cross section view the of the torqueable kink-resistant guidewire 21 along line 19-19 of FIG. 18. The combined FIGS. 21a and 21b are a foreshortened section view along line 21a,21b-21a-21b of FIG. 18, excluding the balloon protector 262, showing the structure of the distal end of the torqueable kink-resistant guidewire 21 including an occlusive balloon 266 and components associatingly connected thereto. With reference to FIGS. 18 and 19 and combined FIGS. 21a and 21b, the torqueable kink-resistant guidewire 21 is now described. Structures are included in the torqueable kink-resistant guidewire 21 which, as opposed to prior art devices, do not include intermediate and bulky joining sleeves, collars and the like. Instead, advantage is taken preferably of taper ground, drawn or otherwise suitably formed tubing sections, swaged joints or other configurations to provide a torqueable kink-resistant guidewire 21 to facilitate a minimum diameter profile, such as 0.014 inch, for purpose of example and illustration. A centrally located inflation tube 268, preferably of nitinol but optionally of other suitable sturdy and flexible material, mountingly accommodates other components thereabout and therealong to partially form the torqueable kink-resistant guidewire 21. The centrally located inflation tube 268 is a tubular structure which can be taper ground having a constant outer diameter central portion 270, a reduced outer diameter proximal end 272 taper ground and which may be reduced to a constant outer diameter being less than the constant outer diameter central portion 270, and a reduced outer diameter distal end 274 taper ground and which may be reduced to a constant diameter being less than the constant diameter central portion 270 and extending through and into the occlusive balloon 266. A lumen 276, shown in dashed lines in FIG. 21a, extends along the length of the centrally located inflation tube 268. A proximally located inflation tube 278, preferably of constant diameter and preferably of stainless steel, is continuous with a smaller and constant diameter crimpable inflation tube 280. The crimpable inflation tube 280 is drawn down from the structure of the proximally located inflation tube 278, whereby a transition area is located between the proximally located inflation tube 278 and the crimpable inflation tube 280. A lumen 282, see FIGS. 18 and 19, extends along and is in common to both the proximally located inflation tube 278 and the crimpable inflation tube 280. The distal end of the proximally located inflation tube 278 is enlarged to provide a receptacle 284 of cylindrical shape for joined and low profile sealed accommodation of the proximal end 272 of the centrally located inflation tube 268 to form a joint 286 between the proximally located inflation tube 278 and the centrally located inflation tube 268. Once the receptacle 284 of the proximally located inflation tube 278 and the proximal end 272 of the centrally located inflation tube 268 are in a position to be joined, the receptacle 284 is three-point swaged to hold the receptacle 284 and the proximal end 272 of the centrally located inflation tube 268 together temporarily. The receptacle 284 is then rotary swaged down onto the proximal end 272 of the centrally located inflation tube 268 until the outer diameter of the joint 286 meets a suitable specification, such as, but not limited to, 0.014 inch. The lumen 282 of the proximally located inflation tube 278 and the crimpable inflation tube 280 connects to and is in communication with the lumen 276 of the centrally located inflation tube 268 as facilitated by the joint 286 which secures the combined proximally located inflation tube 278 and crimpable inflation tube 280 to the centrally located inflation tube 268. An optional transition 267 formed by adhesive can be included between the end of the receptacle 284 and the proximal end 272 of the centrally located inflation tube 268. The distal portion of the centrally located inflation tube 268 serves as a mount for distally located components and features of the torqueable kink-resistant guidewire 21, as shown in combined FIGS. 21a-21b. A floppy tip core 288 is twice shown where in one instance the floppy tip core 288 is shown integrated into the structure of the distal end of the torqueable kink-resistant guidewire 21 and in another instance removed from and distanced from the distal end of the torqueable kink-resistant guidewire 21 and rotated 90° for the purpose of clarity and for a fuller illustrative example of the overall shape thereof. The floppy tip core 288 includes a tapered proximal portion 290, a centrally located flat portion 292, and a distally located flat and widened portion 294. The tapered proximal portion 290 is coaxially aligned within the distal portion of the lumen 276 of the centrally located inflation tube 268 in mutual and frictional engagement, thereby blocking the distal portion of the lumen 276 at the tip 296 at the extreme distal end of the centrally located inflation tube 268. A plurality of inflation orifices 298a-298n are included at the distal end 274 of the centrally located inflation tube 268 in alignment with and for communication with the occlusive balloon 266. A distally located spring coil 302 is wound before assembly, whereby the consecutive coils are impinging upon the previous coil, making the distally located spring coil 302 very tight and stiff and preferably having the same attributes as the proximally located spring coil 220 and the distally located spring coil 238. The inner diameter of the distally located spring coil 302 tightly approximates the outer diameter of the underlying tip 296 of the centrally located inflation tube 268 and allows sufficient room for flexed accommodation of the floppy tip core 288. The distal end of the distally located spring coil 302 and a portion of the flat and widened portion 294 of the floppy tip core 288 are accommodated and connected by a rounded distal tip 304 which is a rounded structure. The distally located spring coil 302 aligns over and about a greater portion of the floppy tip core 288 and a short distance partially along the distal end 274 of the centrally located inflation tube 268 and distal to the inflation orifices 298a-298n where the proximal end of the distally located spring coil 302 is attached to the distal end 274 by an adhesive connection 306. The distal end of a proximally located spring coil 308, preferably having the same attributes as the proximally located spring coil 220, the distally located spring coil 238, and the distally located spring coil 302, aligns over and about the distal end 274 of the centrally located inflation tube 268 proximal to the inflation orifices 298a-298n and is secured to the distal end 274 of the centrally located inflation tube 268 by a adhesive connection 310. The proximal end of the proximally located spring coil 308 secures to the centrally located inflation tube 268 by a adhesive connection 311. The coils at the distal portion of the proximally located spring coil 308 are expanded and opened in order to provide gaps for accommodation of the adhesive connection 310 and to also provide for a stronger bond.

The occlusive balloon 266 is stretchingly mounted generally in the same manner and fashion described for the mounting of the occlusive balloon 224 of the torqueable kink-resistant guidewire 20 where the distal balloon neck 312 and the proximal balloon neck 314 of the occlusive balloon 266 are mounted over and about interceding portions of the distally located coil spring 302 and proximally located spring 308 instead of directly to an inflation tube. The method of balloon bonding is first to bond the distal balloon neck 312 over and about the proximal portion of the distally located spring coil 302 using an adhesive 316 which preferably is a UV cure urethane adhesive ensuring that adhesive 316 is located between the inner bore of the distal balloon neck 312 and the corresponding portion of the open wound portion of the distally located spring coil 302, as well as also extending from the edge of the distal balloon neck 312 a short distance along the distally located spring coil 302. Then a short length of (1.0″ long and ID of 0.037″ for the purpose of example and illustration) tubing fractionally larger than the occlusive balloon 266 and distal balloon neck 312 is slid over and about the cured adhesive 316 and the distal balloon neck 312 at the distal end of the occlusive balloon 266. The proximal balloon neck 314 is pulled gently in a proximal direction as the tube is slid in light frictional engagement in a proximal direction over and about the occlusive balloon 266, thereby stretching the occlusive balloon 266 proximally. Such stretching is continued until the proximal edge of the silicone tube is fully engaged over and about the body of the occlusive balloon 266. This configuration compressingly holds the balloon body down and in the extended and stretched position while the proximal balloon neck 314 is bonded using a similar adhesive 318 ensuring that adhesive 318 is located between the inner bore of the proximal balloon neck 314 and in contact between and along the corresponding portion of the open wound portion of the proximally located spring coil 308, as well as also extending from the edge of the proximal balloon neck 314 a short distance along the proximally located spring coil 308. The tube is subsequently removed. The suitably sized balloon protector 262 having a flared section 264, shown in FIG. 18, with an ID of 0.037″, for example, is slid over the occlusive balloon 266 while rotating the balloon protector 246 so the folds of the occlusive balloon 266 are turned and rolled. When the device is hydrophilically coated, the cure temperature of the coating exposes the entire device to 145° F. which “sets” the occlusive balloon 266 in this orientation so that when the physician uses the device, the profile is well under 0.038″ for example.

### Mode of Operation

Prior to use of the invention, a pressure check (leak test) of the handheld control mechanism 12 is accomplished where such a test very nearly replicates the operation of the invention when incorporating the torqueable kink-resistant guidewire 20 or 21. With the torqueable kink-resistant guidewire 20 or 21 disengaged from the handheld control mechanism 12, the operator:

1. positions the thumb of the left hand on the evacuation control valve actuator button 64;

2. positions the index finger of the right hand in the actuator ring 44 of the evacuation syringe 42;

3. depresses and holds the evacuation control valve actuator button 64 to open the evacuation control valve 63;

4. uses the index finger of the right hand to cycle the actuator ring 44 and attached plunger 43 inwardly and outwardly several times to evacuate the four-port infusion “Y” 51 and appropriate connected tubes, passages and the like until a less than zero ATM is read on the pressure gauge 50;

5. completely releases pressure on the evacuation control valve actuator button 64 to allow closure of the evacuation control valve 63;

6. removes the index finger from the actuator ring 44 to allow free floating of the evacuation syringe 42, while observing the pressure gauge 50 for no change in position;

7. places the index finger of the left hand on and depresses the inflation control valve actuator button 55 to open the inflation control valve 54;

8. grasps and actuates the actuator pad 40 and actuates the plunger 39 of the inflation syringe 38 slowly and inwardly to induce CO₂ and pressurize the four-port infusion “Y” 51 and appropriate connected tubes, passages and the like to 1.5 ATM as read on the pressure gauge 50;

9. releases the inflation control valve actuator button 55 to close the inflation control valve 54 while checking the pressure gauge 50 for stable and maintained pressure; and,

10. resets for inflation by clearing CO₂ and/or air from the four-port infusion “Y” 51 and appropriate connected tubes, passages and the like by depressing the evacuation control valve actuator button 64 to open the evacuation control valve 63, thereby automatically releasing the pressurized gas in the four-port infusion “Y” 51 and appropriate connected tubes, passages and the like, followed by a slight withdrawing actuation of the plunger 43 until the pressure gauge 50 reads zero to depressurize and to expel CO₂ and/or air through the Luer connector purge check valve 130. This resets the vacuum potential of the evacuation syringe 42.

Subsequent to successfully completing the above steps, the torqueable kink-resistant guidewire 20 or 21 operation of the invention is accomplished by joining the handheld control mechanism 12 to the torqueable kink-resistant guidewire 20 or 21 and then advancing the torqueable kink-resistant guidewire 20 or 21 along the vasculature to position the occlusive balloon 224 or 266 just beyond a region of thrombus, plaque, or other undesirable buildup in the vasculature where a thrombectomy may occur, such as with a cross stream thrombectomy catheter, or for placing a stent and/or performing a thrombectomy. Alternatively, the torqueable kink-resistant guidewire 20 or 21 can be advanced to the thrombus site and then connected to the handheld control mechanism 12. Such connection is made by inserting the crimpable inflation tube 155 of the torqueable kink-resistant guidewire 20 or the crimpable inflation tube 280 of the torqueable kink-resistant guidewire 21 into the receptor orifice 48 of the handheld control mechanism 12, whereby the crimpable inflation tube 155 or 280 passes through and within the compression sealing mechanism 49 to communicate with the interior of the male Luer connector 144 for communication with the four-port infusion “Y” 51 and the components connected thereto including, but not limited to, the evacuation syringe 42, the evacuation control valve 63, the inflation syringe 38 and the inflation control valve 54.

Thence, continuing with the mode of operation and with the torqueable kink-resistant guidewire 20 or 21 engaged with the handheld control mechanism 12, and with the occlusive balloon 224 or 266 of the torqueable kink-resistant guidewire 20 or 21 engaged within the vasculature just beyond the thrombus site, the operator:

1. repeats steps 1-7 above to prepare to inflate the occlusive balloon 224 or 266, thereby causing the occlusive balloon 224 or 266 to contact the side of the vasculature to cause a temporary occlusion; performs step 8 except pressurizing to 0.7 ATM (or other pressure indicated by IFU) until vessel is occluded as evidenced by fluoroscopy; and after occlusion, keeps pressure at 0.7 ATM for 15 seconds (or other time indicated by the IFU);

2. firmly depresses the actuator pad 176 of the inflation tube sealing mechanism 47 to pinch and sever the crimpable inflation tube 155 or 280 to cause sealing thereof to maintain pressure therein and in the inflated occlusive balloon 224 or 266, as well as to cause severing of the crimpable inflation tube 155 or 280; and,

3. removes the handheld control mechanism 12 from contact with the pressurized torqueable kink-resistant guidewire 20 or 21 and leaves the torqueable kink-resistant guidewire 20 or 21 having the inflated occlusive balloon 224 or 266 within the vasculature to allow the torqueable kink-resistant guidewire 20 or 21 to be used as an ordinary guidewire where a cross stream thrombectomy catheter may be used for a thrombectomy procedure or may be used to block lysins from passage beyond the temporary occlusion at the inflated occlusive balloon 224 or 266.

Removal of the torqueable kink-resistant guidewire 20 or 21 from the vasculature is facilitated by cutting of the proximal portion of the crimpable inflation tube 155 or 280 with an appropriate cutting tool, such as a scissors which is supplied (not shown), to cause deflation of the occlusive balloon 224 or 266 and by then removing the torqueable kink-resistant guidewire 20 or 21 from the vasculature. The occlusive balloon 224 or 266 can be deflated quicker if the cut crimpable inflation tube 155 or 280 is reinserted into the compression sealing mechanism 49 and vacuum is reestablished. The torqueable kink-resistant guidewire 20 or 21 may then be reused according to the remaining length of the crimpable inflation tube 155 or 280 to provide for one or more temporary occlusions within the vasculature.

Various modifications can be made to the present invention without departing from the apparent scope thereof.

It is claimed:

1. An occlusive guidewire system comprising:
an ergonomic handheld control mechanism prepackaged in a pressurized gaseous environment; and,
a torqueable kink-resistant guidewire having a distally located occlusive balloon, the guidewire with occlusive balloon being prepackaged and compatible with the ergonomic handheld control mechanism.

2. The occlusive guidewire system of claim 1, wherein the prepackaged control mechanism is accompanied by other components included within the prepackaged pressurized gaseous environment for subsequent use in conjunction with the control mechanism, but which other components do not benefit from the pressurized gaseous environment.

3. The occlusive guidewire system of claim 1, wherein the pressurized gaseous environment and the control mechanism packaged therein are contained within a canister body and a canister lid is sealingly attached to the canister body, the canister body and the canister lid sealingly attached to the canister body together defining an airtight and internal pressure resistant package.

4. The occlusive guidewire system of claim 3, wherein the canister lid includes a pull ring, which pull ring allows separation of the canister lid from the canister, so as to release the control mechanism packaged therein.

5. The occlusive guidewire system of claim 3, further including means within the canister body for shock resistant cushioning of the control mechanism.

6. The occlusive guidewire system of claim 5, wherein the means within the canister body for shock resistant cushioning of the control mechanism include a spacer pouch.

7. The occlusive guidewire system of claim 5, wherein the means within the canister body for shock resistant cushioning of the control mechanism include a foam disk in an interior end of the canister.

8. The occlusive guidewire system of claim 5, wherein the means within the canister body for shock resistant cushioning of the control mechanism include a foam disk adjacent to the canister lid.

9. The occlusive guidewire system of claim 5, wherein the means within the canister body for shock resistant cushioning of the control mechanism include a foam disk in an end of the canister, a foam disk adjacent to the canister lid, and a spacer pouch which closely accommodates the interior of the canister between the foam disk in an end of the canister and the foam disk adjacent to the canister lid.

10. The occlusive guidewire system of claim 3, further including a paper bearing written instructions.

11. The occlusive guidewire system of claim 3, wherein the canister body bears a label.

12. The occlusive guidewire system of claim 1, wherein the control mechanism includes:
a housing having an first half and a second half; and,
an internal mechanism assembly at least partially encased within the housing, the internal mechanism assembly including:
an inflation syringe connected to an inline inflation control valve controlled by an actuator button;
an evacuation syringe;
a pressure gauge;
an inflation tube sealing mechanism connected to a compression sealing mechanism; and,
a four-port infusion “Y” connecting, via a first port, the inflation syringe inline control valve, the actuator button and the inflation syringe, via a second port, the evacuation syringe, via a third port the pressure gauge and via a fourth port, the compression sealing mechanism and inflation tube sealing mechanism.

13. The occlusive guidewire system of claim 12, wherein the control mechanism includes an access hole for injection of CO₂.

14. The occlusive guidewire system of claim 12, wherein the inflation tube sealing mechanism includes a multiple guide cell seal.

15. The occlusive guidewire system of claim 14, wherein multiple guide cell seal of the inflation tube sealing mechanism includes geometric shapes to direct or steer multiple crimpable inflation tubes, one at a time, away from each other, thereby decreasing likelihood of puncturing in a touching adjacent relationship.

16. The occlusive guidewire system of claim 1, wherein the pressurized gaseous environment consists of CO₂.

17. The occlusive guidewire system of claim 1, wherein the torqueable kink-resistant guidewire includes:
a balloon, the balloon being inflatable and deflatable; and,
a flexible tip located at or near a distal location on the guidewire.

18. The occlusive guidewire system of claim 1, wherein the torqueable kink-resistant guidewire has a proximal end and a distal end and includes, in proximal to distal connected order:
a crimpable inflation tube;
a coaxially arranged supporting inflation tube and free-floating primary inflation tube;
an inflation tube;
a balloon attached to and aligned over and secured about the inflation tube;
a spring coil aligned over and about the inflation tube proximal to the balloon; and,
a flexible guidewire tip.

19. The occlusive guidewire system of claim 18, wherein the coaxial relationship arrangement of the supporting inflation tube and free-floating primary inflation tube interact to contribute and provide characteristic kink resistance and torqueability by transferring proximal rotational force to the distal section in a one-to-one fashion.

20. The occlusive guidewire system of claim 18, wherein the coaxially arranged supporting inflation tube and free-floating primary inflation tube serve to transfer inflation gas from an inflation structure in the control mechanism to the balloon.

21. The occlusive guidewire system of claim 18, wherein the coaxially arranged supporting inflation tube and the free-floating primary inflation tube are formed of metal, plastic or composite.

22. The occlusive guidewire system of claim 18, wherein the crimpable inflation tube is configured to have crimpable attributes enabling repeated sealing.

23. The occlusive guidewire system of claim 18, wherein the balloon is formed of materials selected from the group consisting of Pellethane 2363 80AE, silicone, Pebax, and polyurethane.

24. The occlusive guidewire system of claim 18, wherein the guidewire is coated with a hydrophilic coating.

25. The occlusive guidewire system of claim 18, wherein the guidewire includes a radiopaque loading material.

26. The occlusive guidewire system of claim 25, wherein the radiopaque loading material is selected from the group consisting of tungsten and BaSO₄.

27. A process of prepackaging an occlusive guidewire system, the process comprising the steps of:
providing an ergonomic handheld control mechanism;
providing an open canister body and a canister lid preassembly;
inserting the ergonomic handheld control mechanism within the open canister body and canister lid preassembly; and,
flushing the internal atmosphere of gases about the inserted ergonomic handheld control mechanism in the preassembly with a desired atmosphere and hermetically sealing the desired atmosphere and inserted ergonomic handheld control mechanism within the preassembly so as to prepackage the ergonomic handheld control mechanism.

28. The process of claim 27, further comprising:
inserting a paper bearing written instructions within the canister body.

29. The process of claim 27, wherein the canister lid has a pull ring for subsequent ease of opening.

30. The process of claim 27, wherein the canister body is labeled to identify the contents.

31. The process of claim 27, wherein the ergonomic handheld control mechanism is rendered resistant to shock by inclusion within the canister body of a spacer pouch, which spacer pouch closely accommodates an exterior profile of the ergonomic handheld control mechanism and which spacer pouch has a periphery which is closely accommodated by an interior of the canister body.

32. The process of claim 27, wherein the ergonomic handheld control mechanism is rendered resistant to shock by inclusion of a foam disk within an interior end of the canister body and a foam disk adjacent to the interior end of the canister lid.

33. The process of claim 27, wherein the ergonomic handheld control mechanism is rendered resistant to shock by inclusion of foam disks in the interior end of the canister body and adjacent to the canister lid, and wherein the ergonomic handheld control mechanism is included within a spacer pouch, which spacer pouch closely accommodates the exterior profile of the ergonomic handheld control mechanism and which spacer pouch has a periphery which is closely accommodated by the interior of the canister body.

34. The process of claim 27, further comprising the step of:
providing a compatible transportation coil with a torqueable kink-resistant guidewire, the coil being sealed within a hermetically sealed pouch.

35. A method of deploying an occlusive guidewire system, the method comprising the steps of:
providing an occlusive balloon and guidewire sealably contained within a transportation coil prepackaged in a pouch;
providing an ergonomic handheld control mechanism in a hermetically sealed container prepackaged in a pressurized gaseous environment, the ergonomic handheld control mechanism being compatible with the occlusive balloon and guidewire;
opening the pouch and opening the sealed container; and,
joining the ergonomic handheld control mechanism to the occlusive balloon and guidewire.

36. The method of claim 35, wherein the sealed container is a canister body and a canister lid.

37. The method of claim 36, wherein the canister lid includes a pull ring, and wherein the step of opening the sealed container includes pulling the pull ring.

38. The method of claim 36, wherein foam disks are provided at an interior end of the canister body and adjacent to the canister lid to provide shock resistance to the ergonomic handheld control mechanism.

39. The method of claims 36, wherein the ergonomic handheld control mechanism is carried in a spacer pouch, which spacer pouch closely accommodates the exterior profile of the ergonomic handheld control mechanism and has a periphery closely accommodated by the interior of the canister body.
